(19) United States
(12) Patent Application Publication
(10) Pub. No.: US 2009/0023727 A1
(43) Pub. Date: Jan. 22, 2009

(54) PHTHALAZINONE DERIVATIVES

(76) Inventors: Muhammad Hashim Javaid, Cambridge (GB); Keith Allan Menear, Cambridge (GB); Niall Morrison Barr Martin, Cambridge (GB); Graeme Cameron Murray Smith, Cambridge (GB); David Alan Rudge, Alderley Park (GB); Craig Anthony Roberts, Alderley Park (GB)

Correspondence Address: MORGAN LEWIS & BOCKIUS LLP, 1111 PENNSYLVANIA AVENUE NW, WASHINGTON, DC, 20004, US

(21) Appl. No.: 12/167,567

(22) Filed: Jul. 3, 2008

Related U.S. Application Data
(60) Provisional application No. 60/948,008, filed on Jul. 5, 2007.
(60) Provisional application No. 61/032,635, filed on Feb. 29, 2008.

(51) Int. Cl. A61K 31/5377; A61K 31/502; C07D 403/08; A61K 31/53; A61P 35/00; C07D 403/14; C07D 413/14

(52) U.S. Cl. 514/234.5; 544/237; 514/248; 544/212; 514/245; 544/116

(57) ABSTRACT

A compound of the formula (I):

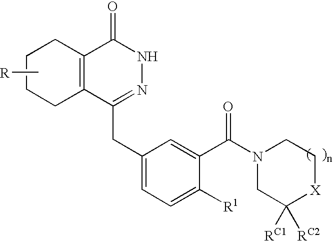

wherein: R represents one or more optional substituents on the fused cyclohexene ring; X can be NR^{X} or CR^{X}R^{Y}; if X=NR^{X} then n is 1 or 2 and if X=CR^{X}R^{Y} then n is 1; if X=NR^{X}, then R^{X} is selected from the group consisting of H, optionally substituted C₁₋₂₀ alkyl, optionally substituted C₅₋₂₀ aryl, optionally substituted C₃₋₂₀ heterocyclyl, optionally substituted amido, optionally substituted thioamido, optionally substituted ester, optionally substituted acyl, and optionally substituted sulfonyl groups; if X=CR^{X}R^{Y} then R^{X} is selected from the group consisting of H, optionally substituted C₁₋₂₀ alkyl, optionally substituted C₅₋₂₀ aryl, optionally substituted C₃₋₂₀ heterocyclyl, optionally substituted amido, optionally substituted thioamido, optionally substituted sulfonamino, optionally substituted ether, optionally substituted ester, optionally substituted acyl, optionally substituted acylamido, and optionally substituted sulfonyl groups and R^{Y} is selected from H, hydroxy, optionally substituted amino, or R^{X} and R^{Y} may together form an optionally substituted spiro-C₃₋₇ cycloalkyl or heterocyclyl group; R^{C1} and R^{C2} are both hydrogen, or when X is CR^{X}R^{Y}, R^{C1}, R^{C2}, R^{X} and R^{Y}, together with the carbon atoms to which they are attached, may form an optionally substituted fused aromatic ring; and R¹ is selected from H and halo.

The present invention relates to phthalazinone derivatives and their use as pharmaceuticals. In particular, the present invention relates to the use of these compounds to inhibit the activity of the enzyme poly (ADP-ribose)polymerase-1, also known as poly(ADP-ribose)synthase and poly ADP-ribosyltransferase, and commonly referred to as PARP-1.

The mammalian enzyme PARP-1 (a 113-kDa multidomain protein) has been implicated in the signalling of DNA damage through its ability to recognize and rapidly bind to DNA single or double strand breaks (D'Amours, et al., Biochem. J., 342, 249-268 (1999)).

The family of Poly (ADP-ribose) polymerases now includes around 18 proteins, that all display a certain level of homology in their catalytic domain but differ in their cellular functions (Ame et al., Bioessays., 26(8), 882-893 (2004)). Of this family PARP-1 (the founding member) and PARP-2 are so far the sole enzymes whose catalytic activity are stimulated by the occurrence of DNA strand breaks, making them unique in the family.

It is now known that PARP-1 participates in a variety of DNA-related functions including gene amplification, cell division, differentiation, apoptosis, DNA base excision repair as well as effects on telomere length and chromosome stability (d'Adda di Fagagna, et al., Nature Gen., 23(1), 76-80 (1999)).

Studies on the mechanism by which PARP-1 modulates DNA repair and other processes has identified its importance in the formation of poly (ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP-1 utilizes NAD⁺ to synthesize poly (ADP-ribose) on a variety of nuclear target proteins, including topoisomerases, histones and PARP itself (Rhun, et al., Biochem. Biophys. Res. Commun., 245, 1-10 (1998))

Poly (ADP-ribosyl)ation has also been associated with malignant transformation. For example, PARP-1 activity is higher in the isolated nuclei of SV40-transformed fibroblasts, while both leukemic and colon cancer cells show higher enzyme activity than the equivalent normal leukocytes and colon mucosa (Miwa, et al., Arch. Biochem. Biophys., 181, 313-321 (1977); Burzio, et al., Proc. Soc. Exp. Biol. Med., 149, 933-938 (1975); and Hirai, et al., Cancer Res., 43, 3441-3446 (1983)). More recently in malignant prostate tumours compared to benign prostate cells significantly increased levels of active PARP (predominantly PARP-1) have been identified associated with higher levels of genetic instability (McNealy, et al., Anticancer Res., 23, 1473-1478 (2003)).

A number of low-molecular-weight inhibitors of PARP-1 have been used to elucidate the functional role of poly (ADP-ribosyl)ation in DNA repair. In cells treated with alkylating agents, the inhibition of PARP leads to a marked increase in DNA-strand breakage and cell killing (Durkacz, et al., Nature, 283, 593-596 (1980); Berger, N. A., Radiation Research, 101, 4-14 (1985)).

Subsequently, such inhibitors have been shown to enhance the effects of radiation response by suppressing the repair of potentially lethal damage (Ben-Hur, et al., British Journal of Cancer, 49 (Suppl. VI), 34-42 (1984); Schlicker, et al., Int. J. Radiat. Bioi., 75, 91-100 (1999)). PARP inhibitors have been reported to be effective in radio sensitising hypoxic tumour cells (U.S. Pat. No. 5,032,617; U.S. Pat. No. 5,215,738 and U.S. Pat. No. 5,041,653). In certain tumour cell lines, chemical inhibition of PARP-1 (and PARP-2) activity is also associated with marked sensitisation to very low doses of radiation (Chalmers, Clin. Oncol., 16(1), 29-39 (2004))

Furthermore, PARP-1 knockout (PARP −/−) animals exhibit genomic instability in response to alkylating agents and γ-irradiation (Wang, et al., Genes Dev., 9, 509-520 (1995); Menissier de Murcia, et al., Proc. Natl. Acad. Sci. USA, 94, 7303-7307 (1997)). More recent data indicates that PARP-1 and PARP-2 possess both overlapping and non-redundant functions in the maintenance of genomic stability, making them both interesting targets (Menissier de Murcia, et al., EMBO. J., 22(9), 2255-2263 (2003)).

PARP inhibition has also recently been reported to have antiangiogenic effects. Where dose dependent reductions of VEGF and basic-fibroblast growth factor (bFGF)-induced proliferation, migration and tube formation in HUVECS has been reported (Rajesh, et al., Biochem. Biophys. Res. Comm., 350, 1056-1062 (2006)).

A role for PARP-1 has also been demonstrated in certain vascular diseases, septic shock, ischaemic injury and neurotoxicity (Cantoni, et al., Biochim. Biophys. Acta, 1014, 1-7 (1989); Szabo, et al., J. Clin. Invest., 100, 723-735 (1997)). Oxygen radical DNA damage that leads to strand breaks in DNA, which are subsequently recognised by PARP-1, is a major contributing factor to such disease states as shown by PARP-1 inhibitor studies (Cosi, et al., J. Neurosci. Res., 39, 38-46 (1994); Said, et al., Proc. Natl. Acad. Sci. U.S.A., 93, 4688-4692 (1996)). More recently, PARP has been demonstrated to play a role in the pathogenesis of haemorrhagic shock (Liaudet, et al., Proc. Natl. Acad. Sci. U.S.A., 97(3), 10203-10208 (2000)), eye (Occular) related oxidative damage as in Macular Degeneration (AMD) and retinitis pigmentosis (Paquet-Durand et al., J. Neuroscience, 27(38), 10311-10319 (2007), as well as in transplant rejection of organs like lung, heart and kidney (O'Valle, et al., Transplant. Proc., 39(7), 2099-2101 (2007). Moreover, treatment with PARP inhibitors has been shown to attenuate acute diseases like pancreatitis and it associated liver and lung damage caused by mechanisms where PARP plays a role (Mota, et al., Br. J. Pharmacol., 151(7), 998-1005 (2007).

It has also been demonstrated that efficient retroviral infection of mammalian cells is blocked by the inhibition of PARP-1 activity. Such inhibition of recombinant retroviral vector infections was shown to occur in various different cell types (Gaken, et al., J. Virology, 70(6), 3992-4000 (1996)). Inhibitors of PARP-1 have thus been developed for the use in anti-viral therapies and in cancer treatment (WO 91/18591).

Moreover, PARP-1 inhibition has been speculated to delay the onset of aging characteristics in human fibroblasts (Rattan and Clark, Biochem. Biophys. Res. Comm., 201(2), 665-672 (1994)) and age related diseases such as atherosclerosis (Hans, et al., Cardiovasc. Res., (Jan. 31, 2008)). This may be related to the role that PARP plays in controlling telomere function (d'Adda di Fagagna, et al., Nature Gen., 23(1), 76-80 (1999)).

PARP inhibitors are also thought to be relevant to the treatment of inflammatory bowel disease (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204), ulcerative colitis (Zingarelli, B, et al., Immunology, 113(4), 509-517 (2004)) and Crohn's disease (Jijon, H. B., et al., Am. J. Physiol. Gastrointest. Liver Physiol., 279, G641-G651 (2000).

Some of the present inventors have previously described (WO 2004/080976) a class of 1(2H)-phthalazinone compounds which act as PARP inhibitors. The compounds have the general formula:

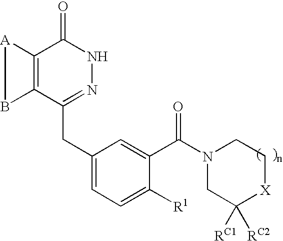

wherein:
A and B together represent an optionally substituted, fused aromatic ring;

### X can be NR^{X} or CR^{X}R^{Y};

if X=NR^{X} then n is 1 or 2 and if X=CR^{X}R^{Y} then n is 1;
R^{X} is selected from the group consisting of H, optionally substituted C₁₋₂₀ alkyl, C₅₋₂₀ aryl, C₃₋₂₀ heterocyclyl, amido, thioamido, sulfonamino, ester, acyl, and sulfonyl groups;
R^{Y} is selected from H, hydroxy, amino;
or R^{X} and R^{Y} may together form a spiro-C₃₋₇ cycloalkyl or heterocyclyl group;
R^{C1} and R^{C2} are both hydrogen, or when X is CR^{X}R^{Y}, R^{C1}, R^{C2}, R^{X} and R^{Y}, together with the carbon atoms to which they are attached, may form an optionally substituted fused aromatic ring; and
R¹ is selected from H and halo.

The present inventors have now discovered that compounds where the fused aromatic ring represented by -A-B- is replaced by a fused cyclohexene ring, the compounds exhibit a surprising increase in the level of inhibition of the activity of PARP, and/or of potentiation of tumour cells to radiotherapy and various chemotherapies, and/or a surprising increase in the solubility of the compound (in aqueous media and/or phosphate buffer solution)—enhanced solubility may be of use in formulation the compounds, for example, for administration by an IV route, or for oral formulations (e.g. liquid and small tablet forms) for paediatric use. The oral bioavailability of the compounds of the present invention may be enhanced. The compounds may also be less susceptible to the action of MDR1 in cells.

Accordingly, the first aspect of the present invention provides a compound of the formula (I):

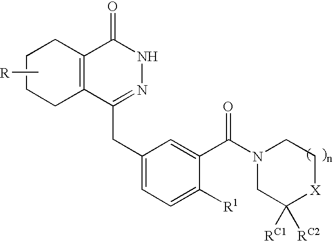

wherein:
R represents one or more optional substituents on the fused cyclohexene ring;

### X can be NR^{X} or CR^{X}R^{Y};

if X=NR^{X} then n is 1 or 2 and if X=CR^{X}R^{Y} then n is 1;
if X=NR^{X}, then R^{X} is selected from the group consisting of H, optionally substituted C₁₋₂₀ alkyl, optionally substituted C₅₋₂₀ aryl, optionally substituted C₃₋₂₀ heterocyclyl, optionally substituted amido, optionally substituted thioamido, optionally substituted ester, optionally substituted acyl, and optionally substituted sulfonyl groups;
if X=CR^{X}R^{Y} then R^{X} is selected from the group consisting of H, optionally substituted C₁₋₂₀ alkyl, optionally substituted C₅₋₂₀ aryl, optionally substituted C₃₋₂₀ heterocyclyl, optionally substituted amido, optionally substituted thioamido, optionally substituted sulfonamino, optionally substituted ether, optionally substituted ester, optionally substituted acyl, optionally substituted acylamido and optionally substituted sulfonyl groups and R^{Y} is selected from H, hydroxy, optionally substituted amino, or R^{X} and R^{Y} may together form an optionally substituted spiro-C₃₋₇ cycloalkyl or heterocyclyl group;
R^{C1} and R^{C2} are both hydrogen, or when X is CR^{X}R^{Y}, R^{C1}, R^{C2}, R^{X} and R^{Y}, together with the carbon atoms to which they are attached, may form an optionally substituted fused aromatic ring; and
R¹ is selected from H and halo.

Therefore, if X is CR^{X}R^{Y}, then n is 1, the compound is of formula (Ia):

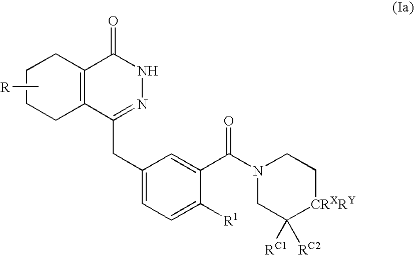

If X is NR^{X}, and n is 1, the compound is of formula (Ib):

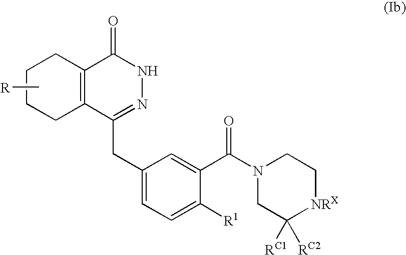

If X is NR^{X}, and n is 2, the compound is of formula (Ic):

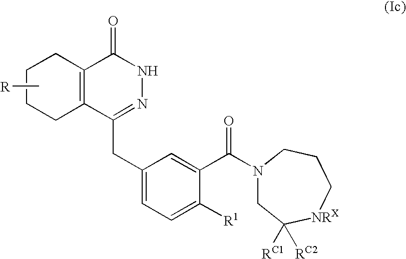

A second aspect of the present invention provides a pharmaceutical composition comprising a compound of the first aspect and a pharmaceutically acceptable carrier or diluent.

A third aspect of the present invention provides the use of a compound of the first aspect in a method of treatment of the human or animal body.

A fourth aspect of the present invention provides the use of a compound as defined in the first aspect of the invention in the preparation of a medicament for:

(a) preventing poly(ADP-ribose) chain formation by inhibiting the activity of cellular PARP (PARP-1 and/or PARP-2);
(b) the treatment of: vascular disease; septic shock; ischaemic injury, both cerebral and cardiovascular; reperfusion injury, both cerebral and cardiovascular; neurotoxicity, including acute and chronic treatments for stroke and Parkinson's disease; haemorraghic shock; eye related oxidative damage; transplant rejection; inflammatory diseases, such as arthritis, inflammatory bowel disease, ulcerative colitis and Crohn's disease; multiple sclerosis; secondary effects of diabetes; as well as the acute treatment of cytoxicity following cardiovascular surgery; pancreatitis; atherosclerosis; or diseases ameliorated by the inhibition of the activity of PARP;
(c) use as an adjunct in cancer therapy or for potentiating tumour cells for treatment with ionizing radiation or chemotherapeutic agents.

In particular, compounds as defined in the first aspect of the invention can be used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. Such combinations could be given, for example, as intravenous preparations or by oral administration as dependent on the preferred method of administration for the particular agent.

Other further aspects of the invention provide for the treatment of disease ameliorated by the inhibition of PARP, comprising administering to a subject in need of treatment a therapeutically-effective amount of a compound as defined in the first aspect, preferably in the form of a pharmaceutical composition and the treatment of cancer, comprising administering to a subject in need of treatment a therapeutically-effective amount of a compound as defined in the first aspect in combination, preferably in the form of a pharmaceutical composition, simultaneously or sequentially with radiotherapy (ionizing radiation) or chemotherapeutic agents.

In further aspects of the present invention, the compounds may be used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, comprising administering to said patient a therapeutically-effective amount of the compound.

The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_{—}000051), RAD51 (NM_{—}002875), RAD51L1 (NM_{—}002877), RAD51C (NM_{—}002876), RAD51L3 (NM_{—}002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE11A (NM_{—}005590) and NBS1 (NM_{—}002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001).

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some preferred embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535) or by an epigenetic mechanism such as gene promoter methylation.

BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol. Med., 8(12), 571-6, (2002)). The association of BRCA1 and/or BRCA2 mutations with breast cancer is well-characterised in the art (Radice, P. J., Exp. Clin. Cancer Res., 21(3 Suppl), 9-12 (2002)). Amplification of the EMSY gene, which encodes a BRCA2 binding factor, is also known to be associated with breast and ovarian cancer.

Carriers of mutations in BRCA1 and/or BRCA2 are also at elevated risk of cancer of the ovary, prostate and pancreas.

In some preferred embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

### DEFINITIONS

The term “aromatic ring” is used herein in the conventional sense to refer to a cyclic aromatic structure, that is, a cyclic structure having delocalised π-electron orbitals.

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C≡CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of cycloalkyl groups include, but are not limited to, those derived from
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);
unsaturated monocyclic hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methyl cyclopentene (C₆), dimethylcyclopentene (C₇), methylcyclohexene (C₇), dimethylcyclohexene (C₈);
saturated polycyclic hydrocarbon compounds:
   thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);
unsaturated polycyclic hydrocarbon compounds:
   camphene (C₁₀), limonene (C₁₀), pinene (C₁₀);
polycyclic hydrocarbon compounds having an aromatic ring:
   indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C₁₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Spiro-C₃₋₇ cycloalkyl or heterocyclyl: The term “spiro C₃₋₇ cycloalkyl or heterocyclyl” as used herein, refers to a C₃₋₇ cycloalkyl or C₃₋₇ heterocyclyl ring joined to another ring by a single atom common to both rings.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The cyclic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

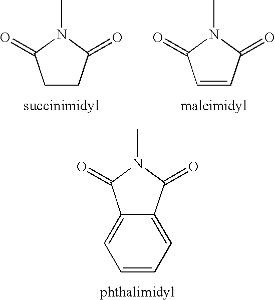

Ureido: —N(R¹)CONR²R³ wherein R² and R³ are independently amino substituents, as defined for amino groups, and R1 is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol: —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

### FURTHER EMBODIMENTS

The following embodiments can apply to each aspect of the present invention, where applicable.

In some embodiments, if X=CR^{X}R^{Y} then R^{X} is selected from the group consisting of H, optionally substituted C₁₋₂₀ alkyl, optionally substituted C₅₋₂₀ aryl, optionally substituted C₃₋₂₀ heterocyclyl, optionally substituted amido, optionally substituted thioamido, optionally substituted sulfonamino, optionally substituted ether, optionally substituted ester, optionally substituted acyl and optionally substituted sulfonyl groups and R^{Y} is selected from H, hydroxy, optionally substituted amino, or R^{X} and R^{Y} may together form an optionally substituted spiro-C₃₋₇ cycloalkyl or heterocyclyl group.

The fused cyclohexene ring may bear one or more substituent groups at any available ring position. These substituents are selected from halo, nitro, hydroxy, ether, thiol, thioether, amino, C₁₋₇ alkyl, C₃₋₂₀ heterocyclyl and C₅₋₂₀ aryl. The fused cyclohexene ring may also bear one or more substituent groups which together form a ring. In particular these may be of formula —(CH₂)ₘ— or —O—(CH₂)ₚ—O—, where m is 2, 3, 4 or 5 and p is 1, 2 or 3. Particular substituents include halo, hydroxy and amino (e.g. NH₂).

If the fused cyclohexene ring bears a sole substituent group, the compound may be of the following formula:

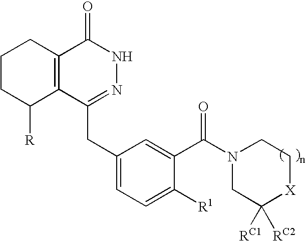

In some embodiments, R¹ is selected from H, Cl and F. In further embodiments, R¹ is F.

In some embodiments, R^{C1} and R^{C2} are both hydrogen.

When n is 2, X is NR^{X}. In these embodiments, R^{X} may be selected from the group consisting of: H; optionally substituted C₁₋₂₀ alkyl; optionally substituted C₅₋₂₀ aryl; optionally substituted ester groups, wherein the ester substituent is preferably C₁₋₂₀ alkyl; optionally substituted acyl groups; optionally substituted amido groups; optionally substituted thioamido groups; and optionally substituted sulfonyl groups. In further embodiments, R^{X} may be selected from the group consisting of: H; optionally substituted C₁₋₂₀ alkyl; optionally substituted C₅₋₂₀ aryl; and optionally substituted ester groups, wherein the ester substituent may be only C₁₋₂₀ alkyl.

When n is 1, X may be NR^{X} or CR^{X}CR^{Y}.

In embodiments where X is NR^{X}, R^{X} may be selected from the group consisting of: H; optionally substituted C₁₋₂₀ alkyl (e.g. optionally substituted C₁₋₇, or C₁₋₄, alkyl); optionally substituted C₅₋₂₀ aryl (e.g. C₅₋₆ aryl); optionally substituted acyl; and optionally substituted sulfonyl. R^{X} may also be selected from optionally substituted ester.

In embodiments where X is NR^{X}, when R^{X} is optionally substituted alkyl, the substituents are may be selected from hydroxy and C₁₋₄ alkoxy (e.g. methoxy). When R^{X} is aryl, it may be heteroaryl (e.g. triazinyl, pyrimidinyl, pyridyl), and in some embodiments may be unsubstituted.

If the aryl group is substituted, the substituents may be selected from C₁₋₄ alkyl (e.g. methyl, trifluoromethyl) and cyano. When R^{X} is optionally substituted acyl, the acyl substituent may be a C₁₋₇ alkyl group (e.g. cyclopropyl) or a C₃₋₂₀, or even C₃₋₇, heterocyclyl group (e.g. tetrahydrofuranyl). When R^{X} is optionally substituted sulfonyl, the sulfone substituent may be a C₁₋₇ alkyl group (e.g. methyl, ethyl, propyl). If R^{X} is ester, the ester group may be C₁₋₄ alkyl (e.g. t-butyl), and may be unsubstituted.

In embodiments where X is CR^{X}R^{Y}, R^{Y} may be H. R^{X} may be selected from the group consisting of: H; optionally substituted C₃₋₂₀ heterocyclyl, more preferably C₃₋₇ heterocyclyl; optionally substituted ether; and optionally substituted sulfonamino. R^{X} may also be optionally substituted amido or optionally substituted acylamido.

In embodiments where X is CR^{X}R^{Y}, when R^{X} is heterocyclyl it may contain one nitrogen ring atom, e.g. pyrrolidinyl. When R^{X} is an ether, the ether substituent may be: C₅₋₇ aryl (e.g. phenyl, pyridyl) which itself may be substituted (for example by chloro or methoxy); C₁₋₇ alkyl (e.g. methyl, ethyl, propyl, butyl, cyclopentyl, cyclopropylethyl), which itself may be substituted by, for example, methoxy. When R^{X} is sulfonamino, the amino substituent may be a C₁₋₇ alkyl group, e.g. methyl, cyclopropyl, and the sulfonamino substituent may be a C₁₋₇ alkyl group (e.g. cyclopropyl) or a C₅₋₇ aryl group, e.g. phenyl, which itself may be substituted (e.g. by chloro). When R^{X} is amido, the first amino substituent may be selected from H and C₁₋₄ alkyl (e.g. methyl), and the second amino substituent may be C₁₋₇ alkyl (e.g. methyl, cyclopropylmethyl, butyl, cyclobutyl), which may itself be substituted by C₅₋₆ aryl (e.g. phenyl) or amino (e.g. dimethylamino). When R^{X} is amido, the amino substituents may together form a ring with the nitrogen atom, such that R^{X} is piperidinylcarbonyl or piperazinylcarbonyl, which may itself be substituted by C₁₋₄ alkyl (e.g. methyl) or sulfonamido (e.g. cyclopropylsulfonylmethylamino). When R^{X} is acylamido, the amide substituent may be H or C₁₋₄ alkyl (e.g. methyl), and the acyl substituent may be C₁₋₇ alkyl (e.g. ethyl) or C₅₋₇ aryl (e.g. phenyl).

In some embodiments, R^{X} is H and R^{Y} is amino. When R^{Y} is amino, the amino substituents may be selected from H and C₁₋₇, or even C₁₋₄, alkyl, such that an amino group may be dimethylamino or the amino substituents may form a ring, such that R^{Y} is, for example, pyrrolidinyl.

Further aspects of the present invention are the compounds of the examples below.

Where appropriate, the above embodiments may be taken in combination with each other.

Compounds of particular interest are those where n is 1, X is CR^{X}R^{Y}, R^{Y} is H and R^{X} is C₁₋₇ alkylether (e.g. methyloxy, ethyloxy, propyloxy, iso-butyloxy, t-butyloxy, cyclopentyloxy, cyclopropylethyloxy), where the C₁₋₇ alkyl group may be substituted, for example, by C₁₋₄ alkoxy (e.g. methoxy). In these embodiments, R¹ may be F and the cyclohexene ring may bear no substituents.

### Includes Other Forms

Included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

### Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Particularly relevant to the present invention is the tautomeric pair illustrated below:

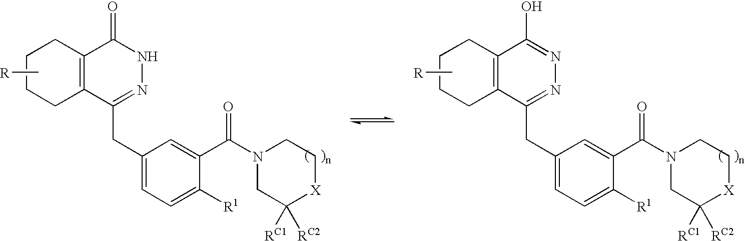

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic and salt forms thereof, for example as discussed below.

Unless otherwise specified, a reference to a particular compound also includes solvates thereof, for example as discussed below.

Unless otherwise specified, a reference to a particular compound also includes prodrugs thereof, for example as discussed below.

Unless otherwise specified, a reference to a particular compound also includes protected forms thereof, for example as discussed below.

Unless otherwise specified, a reference to a particular compound also includes different polymorphic forms thereof, for example as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., “Pharmaceutically Acceptable Salts”, J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO.).

For example, a carboxylic acid group may be protected as an ester for example, as: an C₁₋₇ alkyl ester (e.g. a methyl ester; a t-butyl ester); a C₁₋₇ haloalkyl ester (e.g. a C₁₋₇-trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₂₀ alkyl (e.g. -Me, -Et); C₁₋₇ aminoalkyl (e.g. aminoethyl; 2-(N,N-diethylamino)ethyl; 2-(4-morpholino)ethyl); and acyloxy-C₁₋₇alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleared by phosphotase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

### Synthesis

Compounds of the present invention may be synthesised by reaction of a compound of Formula 1:

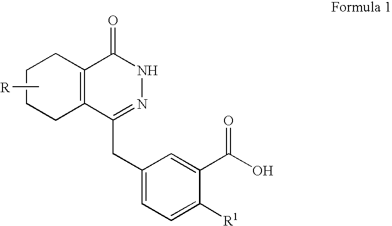

in which R and R¹ are as previously defined, with a compound of Formula 2:

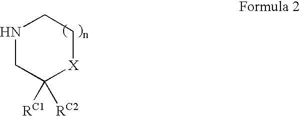

in which n, R^{C1}, R^{C2} and X are as previously defined, in the presence of a coupling reagent system, for example 2-(1H-benzotriazol-1-yl)-1,1,3,3-tetramethyluronium tetrafluoroborate, 2-(1H-benzotriazol-1-yl)-1,1,3,3-tetramethyluronium hexafluorophosphate or (dimethylaminopropyl)ethylcarbodiimide hydrochloride/hydroxybenzotriazole, in the presence of a base, for example diisopropylethylamine, in a solvent, for example dimethylacetamide or dichloromethane, at a temperature in the range of 0° C. to the boiling point of the solvent used.

Alternatively, compounds of the present invention may be synthesised by conversion of a compound of Formula 1 into an activated species, for example an acid chloride or an activated ester such as an N-hydroxysuccinimide ester, using well-known methodologies, and reaction of the activated species with a compound of Formula 2.

Compounds of Formula 1 may be synthesised by reaction of a compound of Formula 3:

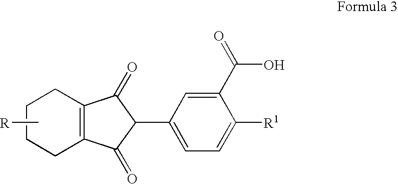

in which R and R¹ are as previously defined, or a compound of Formula 4:

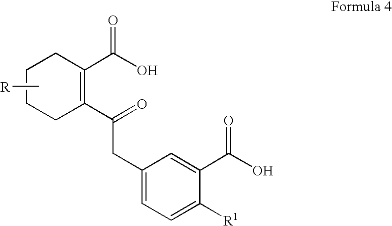

in which R and R¹ are as previously defined, or a mixture of a compound of Formula 3 and a compound of Formula 4, with a source of hydrazine, for example hydrazine hydrate, optionally in the presence of a base, for example triethylamine, optionally in the presence of a solvent, for example industrial methylated spirit, at a temperature in the range of 0° C. to the boiling point of the solvent used.

Compounds of Formula 3 or Formula 4, or mixtures thereof, may be synthesised by reaction of a compound of Formula 5:

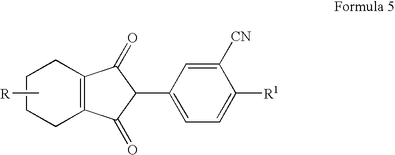

in which R and R¹ are as previously defined, with a reagent capable of hydrolysing a nitrile moiety, for example sodium hydroxide, in the presence of a solvent, for example water, at a temperature in the range of 0° C. to the boiling point of the solvent used.

Compounds of Formula 5 may be synthesised by reaction of a compound of Formula 6:

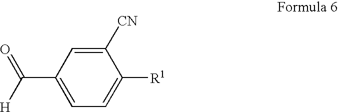

in which R¹ is as previously defined, with a compound of Formula 7:

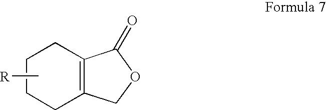

in which R is as previously defined, in the presence of a base, for example sodium methoxide, in a solvent, for example methanol, optionally in the presence of a water scavenger, for example ethyl propionate, at a temperature in the range of 0° C. to the boiling point of the solvent used.

Compounds of Formula 1 may also be synthesised by reaction of a compound of Formula 8:

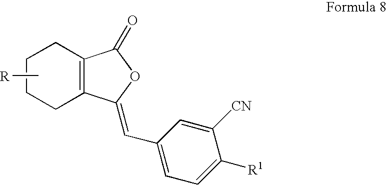

in which R and R¹ are as previously defined, with a reagent capable of hydrolysing a nitrile moiety, for example sodium hydroxide, in the presence of a solvent, for example water, at a temperature in the range of 0° C. to the boiling point of the solvent used, followed by reaction of the resulting intermediate with a source of hydrazine, for example hydrazine hydrate, at a temperature in the range of 0° C. to the boiling point of the solvent used.

Compounds of Formula 8 may be synthesised by reaction of a compound of Formula 9:

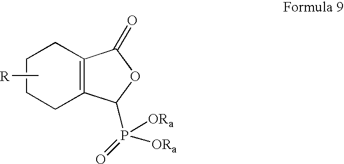

in which R is as previously defined and Rₐ is a C₁₋₄ alkyl group, with a compound of Formula 6, in the presence of a base, for example triethylamine or lithium hexamethyldisilazide, in the presence of a solvent, for example tetrahydrofuran, at a temperature in the range of −80° C. to the boiling point of the solvent used.

Compounds of Formula 9 may be synthesised by methods analogous to those described in WO 02/26576.

Compounds of Formula 1 may also be synthesised by methods analogous to those described above in which the nitrile moiety in all Formulae is replaced by other moieties capable of generating a carboxylic acid, for example ester or carboxamide moieties, or a precursor to the nitrile (e.g. bromo)

Compounds of Formula 2 are commercially available or may be synthesised by methods reported in the chemical literature.

Compounds of the present invention in which X is CR^{X}R^{Y}, in which one of R^{X} or R^{Y} is an amido moiety, and which may therefore be represented by Formula 10:

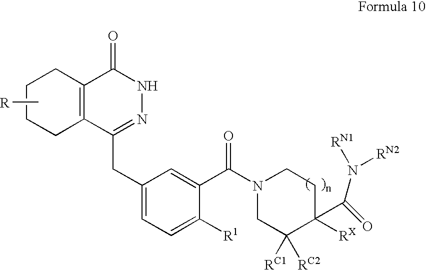

in which R, n, R^{C1}, R^{C2}, R¹ and R^{X} are as previously defined and R^{N1} and R^{N2} are each individually selected from the group consisting of H, optionally substituted C₁₋₂₀ alkyl, C₅₋₂₀ aryl, C₃₋₂₀ heterocyclyl, or may together form an optionally substituted C₃₋₇ cycloalkyl or heterocyclyl group, may be synthesised by reaction of a compound of Formula 11:

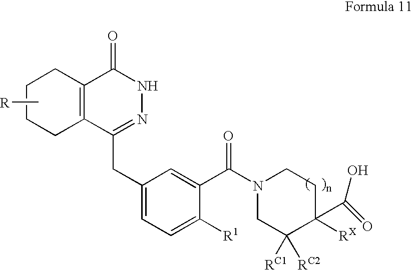

in which R, n, R^{C1}, R^{C2}, R¹ and R^{X} are as previously defined, with a compound of Formula HNR^{N1}R^{N2}, in which R^{N!} and R^{N2} are as previously defined, in the presence of a coupling reagent system, for example 2-(1H-benzotriazol-1-yl)-1,1,3,3-tetramethyluronium tetrafluoroborate, 2-(1H-benzotriazol-1-yl)-1,1,3,3-tetramethyluronium hexafluorophosphate or (dimethylaminopropyl)ethylcarbodiimide hydrochloride/hydroxybenzotriazole, in the presence of a base, for example diisopropylethylamine, in a solvent, for example dimethylacetamide or dichloromethane, at a temperature in the range of 0° C. to the boiling point of the solvent used.

Alternatively, compounds of Formula 10 may be synthesised by conversion of a compound of Formula 11 into an activated species, for example an acid chloride or an activated ester such as an N-hydroxysuccinimide ester, using well-known methodologies, and reaction of the activated species with a compound of Formula HNR^{N1}R^{N2}.

Compounds of Formula 11 may be synthesised by deprotection of a protected form of a compound of Formula 11, for example a compound of Formula 12:

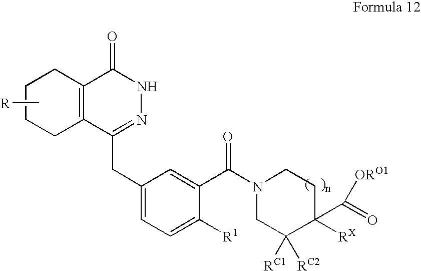

in which R, n, R^{C1}, R^{C2}, R¹ and R^{X} are as previously defined and R⁰¹ is a C₁₋₄ alkyl group, using well known methodologies, for example base-catalysed hydrolysis in the presence of a source of hydroxide, for example sodium or lithium hydroxide, in the presence of a solvent, for example water and/or tetrahydrofuran, at a temperature in the range of 0° C. to the boiling point of the solvent used.

Compounds of Formula 12 may be synthesised from compounds of Formula 1 by the previously described methods.

Compounds of Formula HNR^{N1}R^{N2} are commercially available or may be synthesised by methods reported in the chemical literature.

Compounds of the present invention in which X is NH and which may therefore be represented by Formula 13

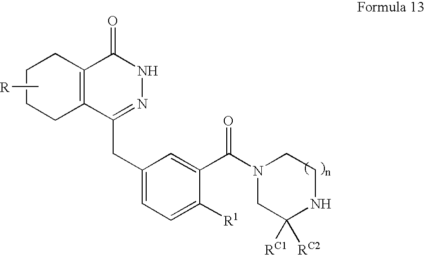

in which R, n, R^{C1}, R^{C2} and R¹ are as previously defined, may be synthesised by deprotection of a protected form of a compound of Formula 13, for example a compound of Formula 14:

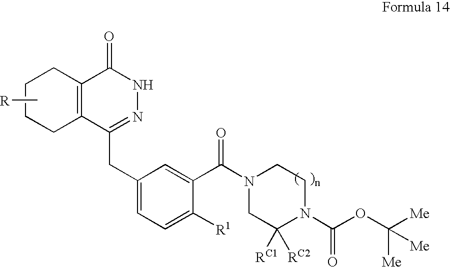

in which n, R^{C1}, R^{C2} and R¹ are as previously defined, using well known methodologies, for example acid-catalysed cleavage, in the presence of an acid, for example trifluoroacetic acid or hydrochloric acid, in the presence of a solvent, for example dichloromethane or ethanol and/or water, at a temperature in the range of 0° C. to the boiling point of the solvent used.

Compounds of Formula 14 may be synthesised from compounds of Formula 1 by the previously described methods.

Compounds of the present invention in which X is NR^{X}, in which R^{X} is an acyl moiety, and which may therefore be represented by Formula 15:

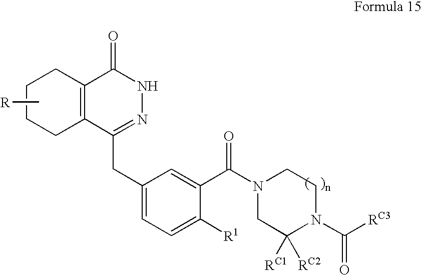

in which R, n, R^{C1}, R^{C2} and R¹ are as previously defined and R^{C3} is selected from the group consisting of optionally substituted C₁₋₂₀ alkyl, C₅₋₂₀ aryl and C₃₋₂₀ heterocyclyl, may be synthesised by reaction of a compound of Formula 13 with a compound of Formula R^{C3}COX, in which R^{C3} is as previously defined and X is a suitable leaving group, for example a halogen such as chloro, optionally in the presence of a base, for example pyridine, triethylamine or diisopropylethylamine, optionally in the presence of a solvent, for example dichloromethane, at a temperature in the range of 0° C. to the boiling point of the solvent used.

Compounds of Formula R^{C3}COX are commercially available or may be synthesised by methods reported in the chemical literature.

Compounds of Formula 15 may also be synthesised by reaction of a compound of Formula 13 with a compound of Formula R^{C3}CO₂H, in which R^{C3} is as previously defined, in the presence of a coupling reagent system, for example 2-(1H-benzotriazol-1-yl)-1,1,3,3-tetramethyluronium tetrafluoroborate, 2-(1H-benzotriazol-1-yl)-1,1,3,3-tetramethyluronium hexafluorophosphate or (dimethylaminopropyl)ethylcarbodiimide hydrochloride/hydroxybenzotriazole, in the presence of a base, for example diisopropylethylamine, in a solvent, for example dimethylacetamide or dichloromethane, at a temperature in the range of 0° C. to the boiling point of the solvent used.

Compounds of Formula R^{C3}CO₂H are commercially available or may be synthesised by methods reported in the chemical literature.

Compounds of the present invention in which X is NR^{X}, in which R^{X} is an amido or thioamido moiety, and which may therefore be represented by Formula 16:

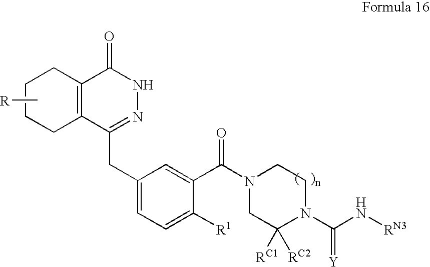

in which R, n, R^{C1}, R^{C2} and R¹ are as previously defined, Y is O or S and R^{N3} is selected from the group consisting of optionally substituted C₁₋₂₀ alkyl, C₅₋₂₀ aryl and C₃₋₂₀ heterocyclyl, may be synthesised by reaction of a compound of Formula 13 with a compound of Formula R^{N3}NCY, in which Y and R^{N3} are as previously defined, in the presence of a solvent, for example dichloromethane, at a temperature in the range of 0° C. to the boiling point of the solvent used.

Compounds of Formula R^{N3}NCY are commercially available or may be synthesised by methods reported in the chemical literature.

Compounds of the present invention in which X is NR^{X}, in which R^{X} is a sulfonyl moiety, and which may therefore be represented by Formula 17:

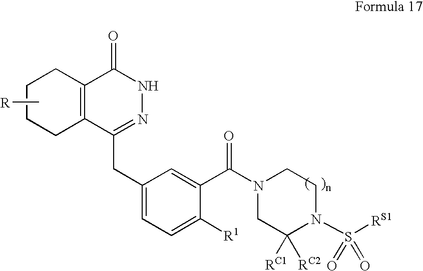

in which R, n, R^{C1}, R^{C2} and R¹ are as previously defined and R^{S1} is selected from the group consisting of optionally substituted C₁₋₂₀ alkyl, C₅₋₂₀ aryl and C₃₋₂₀ heterocyclyl, may be synthesised by reaction of a compound of Formula 13 with a compound of Formula R^{S1}SO₂Cl, in which R^{S1} is as previously defined, optionally in the presence of a base, for example pyridine, triethylamine or diisopropylethylamine, in the presence of a solvent, for example dichloromethane, at a temperature in the range of 0° C. to the boiling point of the solvent used.

Compounds of Formula R^{S1}SO₂Cl are commercially available or may be synthesised by methods reported in the chemical literature.

Compounds of the present invention in which X is NR^{X}, in which R^{X} is selected from the group consisting of optionally substituted C₁₋₂₀ alkyl or C₃₋₂₀ heterocyclyl, and which may therefore be represented by Formula 18:

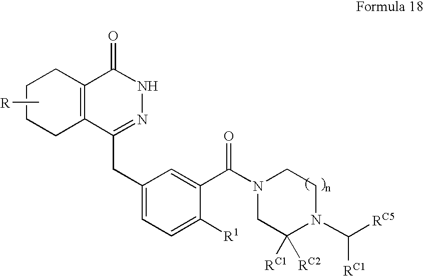

in which R, n, R^{C1}, R^{C2} and R¹ are as previously defined and R^{C4} and R^{C5} are each individually selected from the group consisting of H, optionally substituted C₁₋₂₀ alkyl, C₅₋₂₀ aryl, C₃₋₂₀ heterocyclyl, or may together form an optionally substituted C₃₋₇ cycloalkyl or heterocyclyl group, may be synthesised by reaction of a compound of Formula 13 with a compound of Formula R^{C4}R^{C5}, in which R^{C4} and R^{C5} are as previously defined, in the presence of a reducing agent, for example sodium cyanoborohydride or sodium triacetoxyborohydride, in the presence of a solvent, for example methanol, optionally in the presence of an acid catalyst, for example acetic acid, at a temperature in the range of 0° C. to the boiling point of the solvent used.

Compounds of Formula R^{C4}COR^{C5} are commercially available or may be synthesised by methods reported in the chemical literature.

Compounds of the present invention in which X is CR^{X}R^{Y}, in which R^{X} is optionally substituted sulfonamino and R^{Y} is H may be represented by Formula 19:

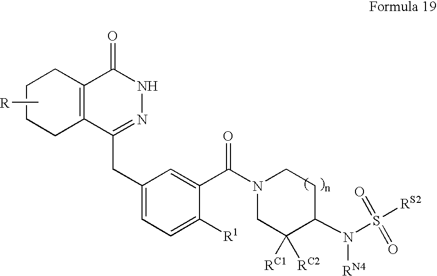

in which R, R^{C1}, R^{C2} and R¹ are as previously defined and R^{N4} is selected from the group consisting of optionally substituted C₁₋₂₀ alkyl, C₅₋₂₀ aryl and C₃₋₂₀ heterocyclyl, and R^{S2} is selected from the group consisting of optionally substituted C₁₋₂₀ alkyl, C₅₋₂₀ aryl and C₃₋₂₀ heterocyclyl, may be synthesised by reaction of a compound of Formula 20:

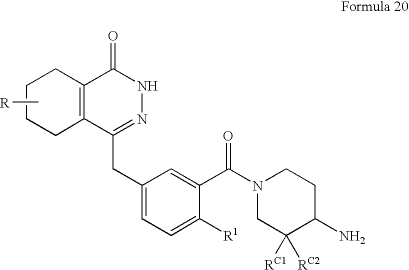

with a compound of Formula R^{S2}SO₂Cl, in which R^{S2} is as previously defined, optionally in the presence of a base, for example pyridine, triethylamine or diisopropylethylamine, in the presence of a solvent, for example dichloromethane, at a temperature in the range of 0° C. to the boiling point of the solvent used. The compound of formula 20 may be synthesized as discussed above.

### Use

The present invention provides active compounds, specifically, active in inhibiting the activity of PARP.

The term “active” as used herein, pertains to compounds which are capable of inhibiting PARP activity, and specifically includes both compounds with intrinsic activity (drugs) as well as prodrugs of such compounds, which prodrugs may themselves exhibit little or no intrinsic activity.

One assay which may conveniently be used in order to assess the PARP inhibition offered by a particular compound is described in the examples below.

The present invention further provides a method of inhibiting the activity of PARP in a cell, comprising contacting said cell with an effective amount of an active compound, preferably in the form of a pharmaceutically acceptable composition. Such a method may be practised in vitro or in vivo.

For example, a sample of cells may be grown in vitro and an active compound brought into contact with said cells, and the effect of the compound on those cells observed. As examples of “effect”, the amount of DNA repair effected in a certain time may be determined. Where the active compound is found to exert an influence on the cells, this may be used as a prognostic or diagnostic marker of the efficacy of the compound in methods of treating a patient carrying cells of the same cellular type.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “adjunct” as used herein relates to the use of active compounds in conjunction with known therapeutic means. Such means include cytotoxic regimes of drugs and/or ionising radiation as used in the treatment of different cancer types. In particular, the active compounds are known to potentiate the actions of a number of cancer chemotherapy treatments, which include the topoisomerase class of poisons (e.g. topotecan, irinotecan, rubitecan), most of the known alkylating agents (e.g. DTIC, temozolamide) and platinum based drugs (e.g. carboplatin, cisplatin) used in treating cancer.

Active compounds may also be used as cell culture additives to inhibit PARP, for example, in order to sensitize cells to known chemotherapeutic agents or ionising radiation treatments in vitro.

Active compounds may also be used as part of an in vitro assay, for example, in order to determine whether a candidate host is likely to benefit from treatment with the compound in question.

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

### Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and 1. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer=s Solution, or Lactated Ringer=s Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

### General Experimental Methods for Examples 1 and 2

### Preparative HPLC

Instrument: Waters ZMD LC-MS system No. LD352 operating in Electrospray ionisation mode.
Mobile Phase A: 0.1% Formic acid in water
Mobile Phase B: 0.1% Formic acid in acetonitrile

### Column: Genesis C18 4 μm 50×4.6 mm

### Gradient:

| Time (mins.) | % B |
|---|---|
| 0 | 5 |
| 7 | 95 |
| 9 | 95 |
| 9.5 | 5 |
| 13 | 5 |

Flow rate: 1.0 ml/min.
PDA Scan range: 210-400 nm.
Alternative Preparative HPLC (Used where Indicated by ⁺)
Instrument: Waters Acquity HPLC/Waters SQD operating in Electrospray ionisation mode.
Mobile Phase A: 0.1% Formic acid in water
Mobile Phase B: 0.1% Formic acid in acetonitrile

### Column: Acquity HPLC BEH C18 1.7 μm 50×2.1 mm

### Gradient:

| Time (mins.) | % B |
|---|---|
| 0 | 5 |
| 0.2 | 5 |
| 2.5 | 95 |
| 3.0 | 95 |

Flow rate: 0.6 ml/min.
PDA Scan range: 210-400 nm.
ELSD Conditions: Drift tube 50 C Nebuliser 20° C. (30%), Gas 50 psi

### Example 1

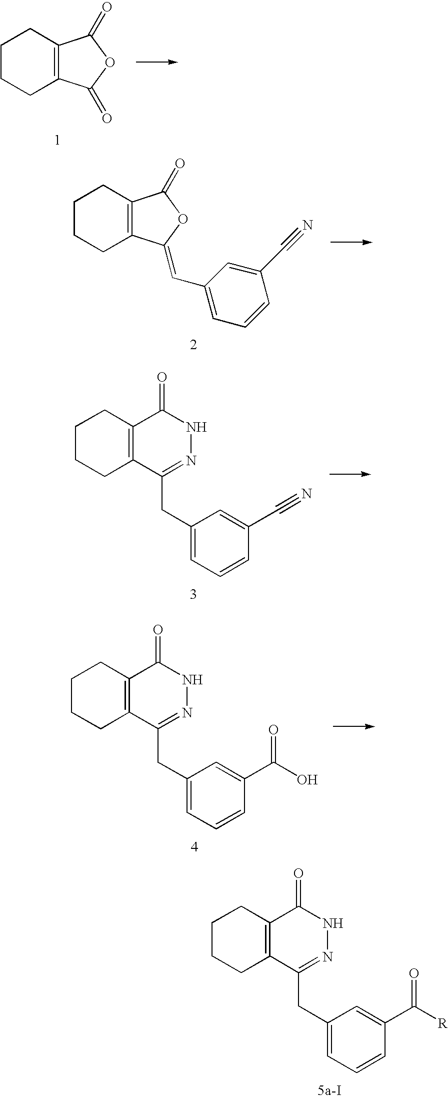

### (a) 3-(3-Oxo-4,5,6,7-tetrahydro-3H-isobenzofuran-1-ylidenemethyl)-benzonitrile (2)

4,5,6,7-Tetrahydro-isobenzofuran-1,3-dione (1) (3.043 g, 20.0 mmol) and 3-cyano phenyl acetic acid (3.15 g, 19.8 mmol) were heated in the presence of sodium acetate (20.1 mg, 0.243 mmol) to 240° C. using a ‘Wood's Alloy’ bath. Once the reaction had reached 240° C. an additional amount of sodium acetate (20.1 mg, 0.243 mmol) was added. The reaction mixture was then heated for a further 40 minutes and then cooled to 80° C. Ethanol (20 ml) was added to the thick gum and the mixture slurried for 30 minutes. The resulting suspension was cooled to ambient temperature and filtered. The solid was further washed with additional cold ethanol (2×4 ml) and dried to afford the desired product as a mixture of geometric isomers. Main peak in LC-MS, (3.5 g, 94% purity) and required no further purification; m/z (LC-MS, ESP), RT=4.75 mins (no ionization observed).

### (b) 3-(4-Oxo-3,4,5,6,7,8-hexahydro-phthalazin-1-ylmethyl)-benzonitrile (3)

A suspension of 3-(3-oxo-4,5,6,7-tetrahydro-3H-isobenzofuran-1-ylidenemethyl)-benzonitrile (2) (3.5 g, 13.9 mmol) in water (20 ml), was treated with hydrazine hydrate (1.0 ml, 20.0 mmol) dropwise and then heated to reflux for 8 hours. The mixture was cooled to approximately 5° C. and the resultant suspension filtered and washed with water (4 ml) and diethyl ether (4 ml). The material was then dried in a vacuo. Main peak in LC-MS, (1.8 g, 91% purity) and required no further purification; m/z (LC-MS, ESP), RT=3.24 mins (M+H 266).

### (c) 3-(4-Oxo-3,4,5,6,7,8-hexahydro-phthalazin-1-ylmethyl)-benzoic acid (4)

To a suspension of 3-(4-oxo-3,4,5,6,7,8-hexahydro-phthalazin-1-ylmethyl)-benzonitrile (3) (1.31 g, 4.93 mmol) in water (10 ml) was added sodium hydroxide (987 mg, 24.7 mmol), and heated for 4 hours at 90° C. The mixture was then cooled and the pH adjusted with sulfuric acid to 2 (ca 6 ml 4N). A cream precipitate resulted which was isolated by filtration and dried. Single peak in LC-MS, (1.1 g, 99% purity) and required no further purification; m/z (LC-MS, ESN), RT=3.10 mins (M+H 283.4).

### (d) Library Synthesis (5a-h)

To a solution of 3-(4-oxo-3,4,5,6,7,8-hexahydro-phthalazin-1-ylmethyl)-benzoic acid (4) (20 mg, 0.07 mmol), in DCM (1 ml) was added HBTU (53 mg, 0.140 mmol), triethylamine (20 μL, 0.140 mol) and amine (0.140 mmol). The reaction mixture was stirred for 18 hours at room temperature and concentrated in vacuo. The crude samples were submitted for preparative HPLC purification.

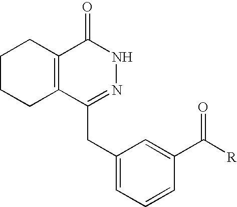

| | | Puri- | RT | |
|---|---|---|---|---|
| | R | ty | (min) | M + H |
| 5a | 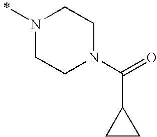 | 97 | 7.58 | 421.1 |
| 5b | 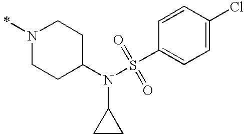 | 99 | 11.93 | 581.0 |
| 5c | 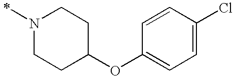 | 99 | 12.32 | 478.0 |
| 5d | 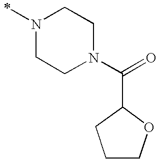 | 90 | 7.21 | 451.0 |
| 5e | 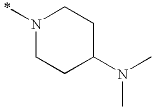 | 93 | 5.39 | 395.1 |
| 5f | 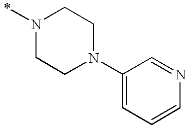 | 87 | 5.94 | 430.1 |
| 5g | 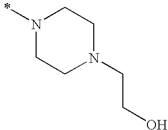 | 85 | 5.10 | 397.1 |
| 5h | 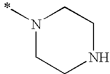 | 85 | 5.04 | 353.2 |
| 5i | 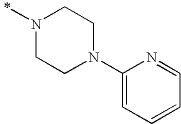 | 99 | 3.71 | 430.3 |
| 5j | 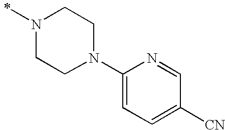 | 100 | 4.66 | 451.3 |
| 5k | 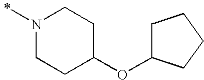 | 94 | 1.88 | 436.4 |
| 5l | 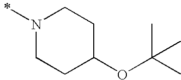 | 95 | 1.78 | 424.4 |

### Example 2

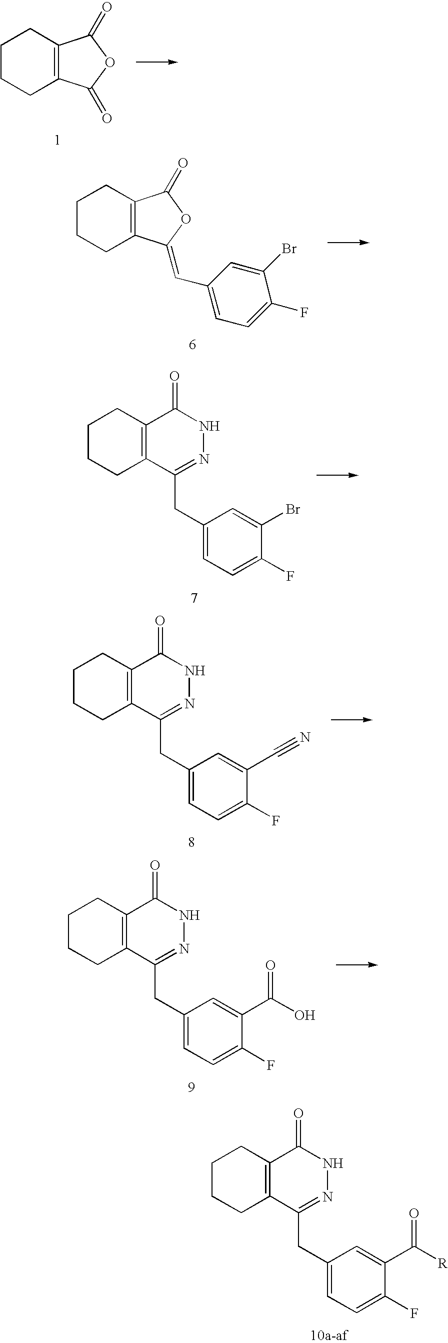

### (a) 3-(3-Bromo-4-fluoro-benzylidene)-4,5,6,7-tetrahydro-3H-isobenzofuran-1-one (6)

4,5,6,7-tetrahydro-isobenzofuran-1,3-dione (1) (16.7 g, 109.7 mmol) and 3-bromo-4-fluorophenylacetic acid (15.0 g, 64.37 mmol) were heated in the presence of sodium acetate (0.259 g, 3.160 mmol) to 210° C. using a ‘Wood's Alloy’ bath for 4.5 hours. The reaction mixture was then poured into a crucible and cooled to give a crystalline solid. The solid was ground with a mortar and pestle and triturated with ethanol (20 ml). The resultant suspension was then filtered and washed with further ethanol (10 ml). The solid was then dried to afford the desired product as a mixture of geometric isomers. Main peak in LC-MS, (20.78 g, 94% purity) and required no further purification; m/z (LC-MS, ESP), RT=4.74 mins (no ionization observed).

### (b) 4-(3-Bromo-4-fluoro-benzyl)-5,6,7,8-tetrahydro-2H-phthalazin-1-one (7)

To 3-(3-bromo-4-fluoro-benzylidene)-4,5,6,7-tetrahydro-3H-isobenzofuran-1-one (6) (cis/trans mixture) (20.78 g, 64.3 mmol) suspended in water (150 ml) was added hydrazine hydrate (12.5 ml, 257.2 mmol). The reaction was heated to 85° C. for 18 hours and then cooled to room temperature. A beige suspension was isolated by filtration and washed with water (1×50 ml), hexane (1×50 ml), and ether (1×25 ml) before being dried overnight in a vacuum oven. Main peak in LC-MS, (19.1 g, 91% purity) and required no further purification; m/z (LC-MS, ESP), RT=3.92 mins (M+H 337 & 339).

### (c) 2-Fluoro-5-(4-oxo-3,4,5,6,7,8-hexahydro-phthalazin-1-ylmethyl)-benzonitrile (8)

To a solution of 4-(3-bromo-4-fluoro-benzyl)-5,6,7,8-tetrahydro-2H-phthalazin-1-one (7) (9.53 g, 28.2 mmol), in dry DMF (95 ml) was added copper (I) cyanide (3.5 g, 42.3 mmol) in one portion. The mixture was heated to 160° C. for 18 hours. The reaction was then cooled and filtered through celite and washed though with methanol (30 ml). The filtrate was concentrated in vacuo to afford a brown oil. Main peak in LC-MS, (8.01 g, 66% purity) and was taken through crude to the next transformation; m/z (LC-MS, ESP), RT=3.50 mins (M+H 284.3).

### (d) 2-Fluoro-5-(4-oxo-3,4,5,6,7,8-hexahydro-phthalazin-1-ylmethyl)-benzoic acid (9)

Crude 2-fluoro-5-(4-oxo-3,4,5,6,7,8-hexahydrophthalazin-1-ylmethyl)benzonitrile (9.9 g, 34.9 mmol) was suspended in water (245 ml) and treated with sodium hydroxide (6.98 g, 174 mmol). The mixture was heated to 60° C. for 18 hours. The reaction was then cooled to 5° C. and concentrated sulfuric acid added dropwise until a precipitate formed (ca 10 ml, pH2). The suspension was stirred for 10 minutes at 5° C. and filtered. The solid isolated was washed with water (2×8 ml) and triturated with DCM (20 ml) before being dried. Single peak in LC-MS, (4.48 g, 98% purity) and was taken through to the next without any further purification; m/z (LC-MS, ESN), RT=1.96 mins (M−H 301.3).

### (e) Library Synthesis (10a-m)

To a solution of 2-fluoro-5-(4-oxo-3,4,5,6,7,8-hexahydro-phthalazin-1-ylmethyl)-benzoic acid (22 mg, 0.07 mmol), in DMA (1 ml) was added HBTU (53 mg, 0.140 mmol), triethylamine (20 μL, 0.140 mol) and amine (0.140 mmol). The crude reaction mixture was stirred for 18 hours at room temperature and then submitted for preparative HPLC purification.

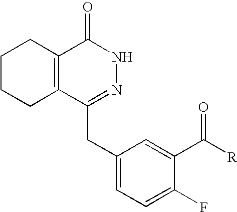

| | | Puri- | RT | M + |
|---|---|---|---|---|
| | R | ty | (min) | H |
| 10a | 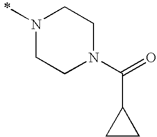 | 100 | 4.19 | 439.0 |
| 10b | 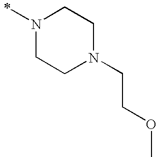 | 99 | 3.51 | 429.1 |
| 10c | 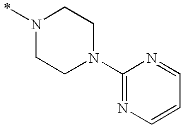 | 98 | 4.50 | 449.0 |
| 10d | 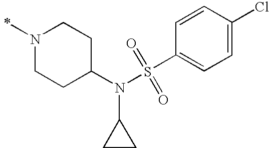 | 99 | 5.50 | 599.1 |
| 10e | 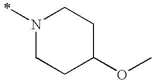 | 99 | 4.34 | 400.1 |
| 10f | 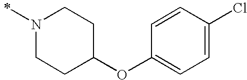 | 100 | 5.66 | 496.0 |
| 10g | 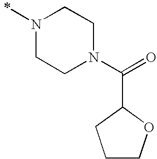 | 95 | 4.05 | 469.1 |
| 10h | 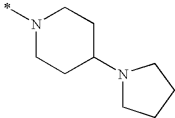 | 99 | 3.54 | 439.1 |
| 10i | 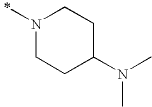 | 99 | 3.49 | 413.1 |
| 10j | 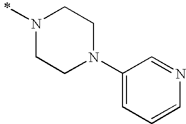 | 97 | 3.64 | 448.1 |
| 10k | 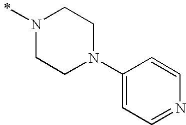 | 98 | 3.62 | 448.1 |
| 10l | 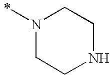 | 82 | 6.89 | 371.2 |
| 10m | 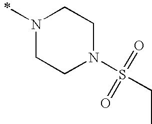 | 92 | 8.39 | 463.2 |
| 10o | 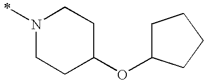 | 90 | 1.93⁺ | 454.4 |
| 10p | 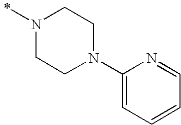 | 100 | 3.56⁺ | 448.3 |
| 10q | 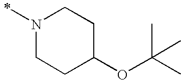 | 95 | 1.78⁺ | 424.4 |
| 10r | 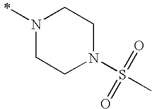 | 92 | 4.24 | 449.2 |
| 10s | 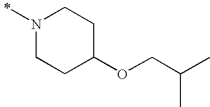 | 94 | 5.40 | 442.2 |
| 10t | 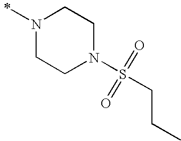 | 91 | 9.20 | 477.2 |
| 10u | 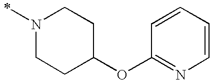 | 99 | 4.89 | 463.2 |
| 10v | 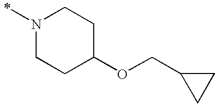 | 92 | 4.74 | 440.2 |
| 10w | 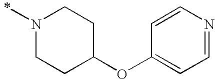 | 99 | 3.79 | 463.2 |
| 10x | 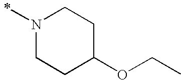 | 100 | 4.69 | 414.2 |
| 10y | 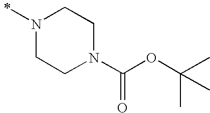 | 100 | 4.82 | 471.4 |
| 10z | 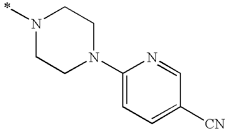 | 100 | 4.65 | 455.3 |
| 10aa | 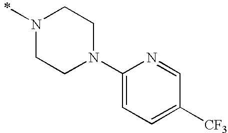 | 100 | 5.41 | 16.3 |
| 10ab | 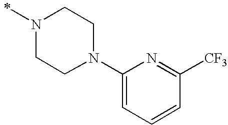 | 98 | 11.52 | 516.2 |
| 10ac | 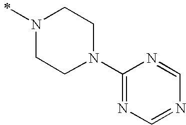 | 100 | 4.14 | 450.3 |
| 10ad | 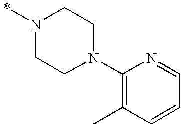 | 98 | 7.33 | 462.3 |
| 10ae | 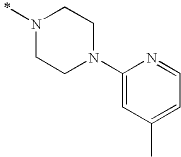 | 100 | 3.74 | 462.3 |
| 10af | 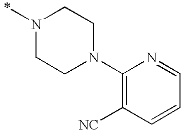 | 96 | 9.86 | 473.3 |

### General Experimental Methods for Examples 3-8

### Analytical LC-MS

LC-MS data was generated on a system where the HPLC component comprised generally either an Agilent 1100, Waters Alliance HT (2790 & 2795) equipment or an HP1100 pump and Diode Array with CTC autosampler and was run on a Phenomenex Gemini C18 5 mm, 50×2 mm column (or similar) eluting with either acidic eluent (for example, using a gradient, over 4 minutes, between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient, over 4 minutes, between 0-95% water/acetonitrile with 5% of a 0.1% 880 Ammonia in acetonitrile mixture); and the MS component comprised generally a Waters ZQ mass spectrometer scanning over an appropriate mass range. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is the (M+H)⁺ for positive ion mode and (M−H)− for negative ion mode

### NMR Spectra

Where given NMR data was determined at 400 MHz using, for example, a Bruker DPX-400 spectrometer and is in the form of delta values, for major diagnostic protons, given in parts per million (ppm). Solvents used were CDCl₃ (with tetramethylsilane (TMS) as an internal standard) or DMSO-d₆ unless otherwise indicated; the following abbreviations have been used: s, singlet; d, doublet; t, triplet; q, quartet; m, multiplet; br, broad.

### Example 3

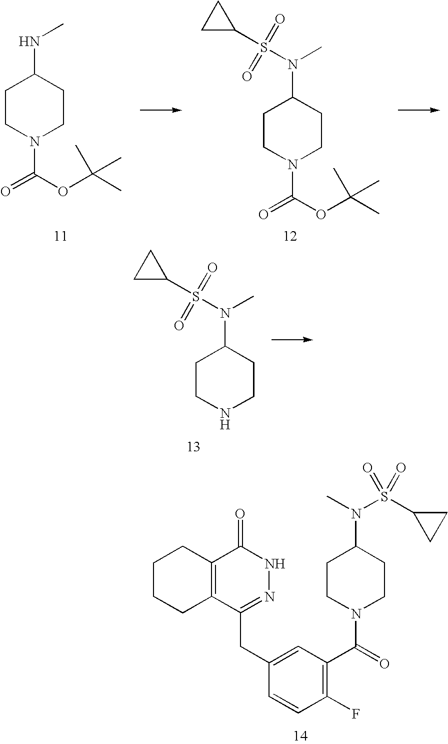

### (a) Tert-butyl 4-(N-methylcyclopropanesulfonamido)piperidine-1-carboxylate (12)

To a solution of tert-butyl 4-(methylamino)piperidine-1-carboxylate (11) (2 g, 9.33 mmol) in dichloromethane (40 ml) was added triethylamine (2.60 ml, 18.67 mmol). Cyclopropanesulfonyl chloride (1.188 ml, 11.67 mmol) was then added dropwise over a period of 2 minutes. The resulting solution was stirred at ambient temperature for 20 hours. Sat. aq. sodium bicarbonate (˜50 mL) was then added and mixture stirred for 5 minutes. The organic layer was then separated, dried over magnesium sulfate, filtered and dried to afford crude desired product (3.40 g, >100%) as an amber oil which solidified on standing; ¹H NMR (400.132 MHz, CDCl3) δ 0.95-1.00 (2H, m), 1.17-1.21 (2H, m), 1.32-1.38 (1H, m), 1.47 (9H, s), 1.58-1.77 (3H, m), 2.26-2.32 (1H, m), 2.71-2.80 (2H, m), 2.81 (3H, s), 3.83-3.91 (1H, m), 4.17-4.26 (2H, m). This was used without further purification, assuming 100% yield.

### (b) N-methyl-N-(piperidin-4-yl)cyclopropanesulfonamide (13)

A solution of tert-butyl 4-(N-methylcyclopropanesulfonamido)piperidine-1-carboxylate (12) (2.96 g, 9.3 mmol) in dichloromethane (20 mL) was treated with trifluoroacetic acid (7.16 mL, 93.00 mmol). The resulting solution was stirred at ambient temperature for 4 hours then poured directly onto an SCX-2 column (50 g). The cartridge was eluted through sequentially with DCM (200 mL) and methanol (150 mL) before the desired product was eluted from the column, using 2M NH₃/MeOH (200 mL), and evaporated to dryness to the desired compound as a waxy yellow solid (1.800 g, 89%); ¹H NMR (400.132 MHz, DMSO) δ 0.91-0.96 (4H, m), 1.55-1.64 (4H, m), 2.44-2.52 (2H, m), 2.55-2.62 (1H, m), 2.72 (3H, s), 2.94-3.00 (2H, m), 3.55-3.65 (1H, m).

### (c) N-(1-(2-fluoro-5-((4-oxo-3,4,5,6,7,8-hexahydrophthalazin-1-yl)methyl)benzoyl)piperidin-4-yl)-N-methylcyclopropanesulfonamide (14)

A solution of 2-fluoro-5-((4-oxo-3,4,5,6,7,8-hexahydrophthalazin-1-yl)methyl)benzoic acid (9) (200 mg, 0.66 mmol) in N,N-dimethylacetamide (6 ml) was treated with triethylamine (0.250 ml, 1.79 mmol) and N-methyl-N-(piperidin-4-yl)cyclopropanesulfonamide (13) (150 mg, 0.69 mmol). O-Benzotriazol-1-yl-N,N,N′,N′-tetra-methyluronium hexafluorophosphate (344 mg, 0.91 mmol) was then added and the reaction mixture was stirred, at ambient temperature, under nitrogen for 6 hours. The reaction mixture was then poured into water (50 mL) and resultant solid filtered to afford crude product as a sticky dark brown solid. The filtrate was adjusted to pH 4-5 by addition of 2M HCl and extracted with DCM (2×75 mL). The combined extracts were combined with the filtered solid from above and mixture washed with brine, dried over magnesium sulfate, filtered and evaporated to afford the crude product, which was purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were combined before being evaporated to dryness and lyophilised to afford the product as a gum. This was redissolved in a minimum amount of dichloromethane, allowed to evaporate on standing and dried under vacuum, at 65° C., for 4 hours to afford the desired compound as a tan foam (128 mg, 38.5% yield, 100% purity by LC-MS); ¹H NMR (399.902 MHz, DMSO) δ 0.96 (4H, d), 1.54-1.80 (8H, m), 2.35-2.40 (4H, m), 2.60-2.66 (1H, m), 2.73 (3H, s), 2.80-2.91 (1H, m), 3.11-3.20 (1H, m), 3.36-3.42 (1H, m), 3.84-3.93 (1H, m), 3.93 (2H, s), 4.56-4.62 (1H, m), 7.19-7.33 (3H, m), 12.62 (1H, s); m/z (LC-MS, ESI+), RT=1.70 (M+H 503.5).

### Example 4

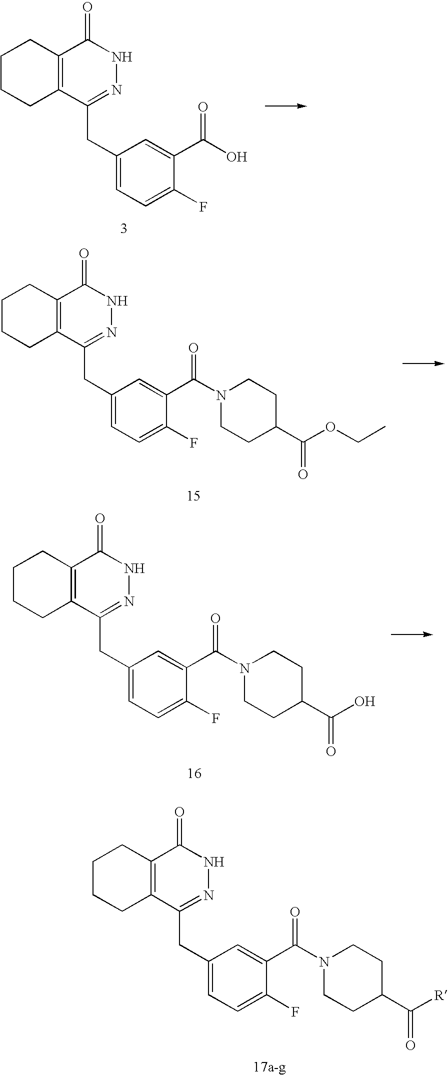

### (a) Ethyl 1-(2-fluoro-5-((4-oxo-3,4,5,6,7,8-hexahydrophthalazin-1-yl)methyl)benzoyl)piperidine-4-carboxylate (15)

A partial solution of 2-fluoro-5-((4-oxo-3,4,5,6,7,8-hexahydrophthalazin-1-yl)methyl)benzoic acid (9) (3 g, 9.92 mmol) in N,N-dimethylacetamide (90 ml) was treated with ethyl isonipecotate (1.9 ml, 12.34 mmol) and triethylamine (3.5 ml, 25.11 mmol). O-Benzotriazol-1-yl-N,N,N′,N′-tetra-methyluronium hexafluorophosphate (4.89 g, 12.90 mmol) was then added portionwise over 5 minutes. Reaction mixture was then stirred at ambient temperature under nitrogen overnight, before being poured into water (˜500 mL). The pH of the mixture was adjusted from pH11-12 to pH 7 by dropwise addition of 2M HCl. The resultant solid was collected by suction filtration to give crude product as a brown sticky gum, which was redissolved in DCM (˜200 mL), washed with brine, dried over magnesium sulfate and evaporated to a brown oil/gum. The filtrate was also extracted with DCM (500 mL) and organic extract dried over magnesium sulfate and evaporated to a dark amber gum. Both crude products were combined and purified by flash silica chromatography, elution gradient 0 to 20% MeOH in DCM. Product containing fractions were evaporated to dryness and re-purified by flash silica chromatography, elution gradient 0 to 10% MeOH in EtOAc. Pure fractions were evaporated to dryness to afford the desired compound as a pale yellow gum (1.900 g, 43.4%); ¹H NMR (400.132 MHz, CDCl3) δ 1.26 (3H, t), 1.66-1.89 (7H, m), 2.00-2.06 (1H, m), 2.33-2.40 (2H, m), 2.52-2.61 (3H, m), 3.03-3.16 (2H, m), 3.51-3.58 (1H, m), 3.88 (2H, s), 4.16 (2H, q), 4.49-4.55 (1H, m), 7.03 (1H, t), 7.17-7.21 (2H, m), 10.64 (1H, s); m/z (LC-MS, ESI+), RT=1.92 (M+H 442.5).

### (b) 1-(2-Fluoro-5-((4-oxo-3,4,5,6,7,8-hexahydrophthalazin-1-yl)methyl)benzoyl)piperidine-4-carboxylic acid (16)

A solution of ethyl 1-(2-fluoro-5-((4-oxo-3,4,5,6,7,8-hexahydrophthalazin-1-yl)methyl)benzoyl)piperidine-4-carboxylate (15) (1.9 g, 4.30 mmol) in ethanol (30 mL) was treated with a solution of lithium hydroxide monohydrate (0.397 g, 9.47 mmol) in water (7.50 mL). The resulting solution was stirred at ambient temperature for 19 hours. The resulting mixture was evaporated to dryness and the residue was redissolved in water (50 mL), washed with DCM (˜20 mL) and the aqueous solution adjusted to pH3, with stirring, by dropwise addition of 2M HCl. The resultant precipitate was collected by suction filtration and dried, under vacuum, at 60° C., for 2 hours to afford the desired compound as a tan solid (1.000 g, 56.2%); ¹H NMR (400.132 MHz, CDCl3) δ 1.66-1.92 (7H, m), 2.05-2.13 (1H, m), 2.29-2.69 (5H, m), 3.10-3.18 (2H, m), 3.54-3.60 (1H, m), 3.86-3.96 (2H, m), 4.45-4.52 (1H, m), 7.01-7.12 (2H, m), 7.21-7.26 (1H, m), 12.58-12.98 (1H, brs) [OH assumed absent/exchanged]; m/z (LC-MS, ESI+), RT=0.82 (M+H 414.5).

### (c) Library Synthesis

1-(2-fluoro-5-((4-oxo-3,4,5,6,7,8-hexahydrophthalazin-1-yl)methyl)benzoyl)piperidine-4-carboxylic acid (16) (896 mg, 2.17 mmol) was dissolved in N,N-dimethylacetamide (18 mL) and solution treated with triethylamine (0.8 mL, 5.74 mmol) and O-Benzotriazol-1-yl-N,N,N′,N′-tetra-methyluronium hexafluorophosphate (1.1 g, 2.90 mmol). The resultant yellow solution was stirred at ambient temperature for 25 minutes to give a stock solution. To each of the desired amines (0.41-0.46 mmol) was added 2.35 mL of the stock solution and the reaction mixtures stirred at ambient temperature overnight. The crude reaction mixtures were filtered before being purified by preparative HPLC (Waters XBridge Prep C18 OBD column, 5μ silica, 19 mm diameter, 100 mm length), using decreasingly polar mixtures of water (containing 1% NH₃) and MeCN as eluents. Fractions containing the desired compounds were evaporated to dryness, lyophilised and dried under high vacuum to afford the desired compounds.

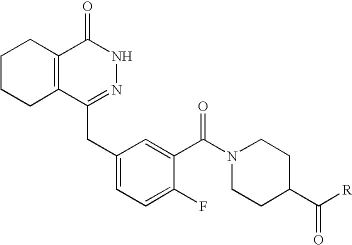

| | R | Purity | RT (min) | M + H |
|---|---|---|---|---|
| 17a | 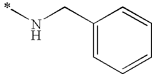 | 100 | 1.81 | 503.5 |
| 17b | 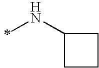 | 96.5 | 1.61 | 467.5 |
| 17c | 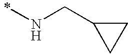 | 100 | 1.57 | 467.5 |
| 17d | 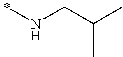 | 100 | 1.66 | 469.5 |
| 17e | 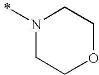 | 100 | 1.4 | 483.4 |
| 17f | 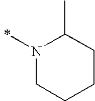 | 100 | 1.83 | 495.5 |
| 17g | 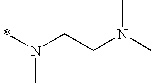 | 100 | 1.44 | 498.5 |
| 17h | 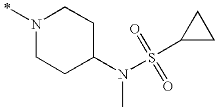 | 100 | 1.68 | 614.5 |

17a: —N-benzyl-1-[2-fluoro-5-[(4-oxo-5,6,7,8-tetrahydro-3H-phthalazin-1-yl)methyl]benzoyl]piperidine-4-carboxamide; ¹H NMR (399.902 MHz, DMSO) δ 1.36-1.65 (7H, m), 1.73-1.80 (1H, m), 2.28-2.33 (4H, m), 2.72-2.84 (2H, m), 2.93-3.03 (1H, m), 3.31-3.37 (1H, m), 3.85 (2H, s), 4.14-4.25 (2H, m), 4.38-4.44 (1H, m), 7.09-7.34 (8H, m), 8.28 (1H, t), 12.53 (1H, s).

17b: —N-cyclobutyl-1-[2-fluoro-5-[(4-oxo-5,6,7,8-tetrahydro-3H-phthalazin-1-yl)methyl]benzoyl]piperidine-4-carboxamide; ¹H NMR (399.902 MHz, DMSO) δ 1.38-1.53 (2H, m), 1.58-1.66 (7H, m), 1.73-1.79 (1H, m), 1.81-1.91 (2H, m), 2.09-2.18 (2H, m), 2.33-2.42 (4H, m), 2.76-2.90 (2H, m), 2.98-3.08 (1H, m), 3.36-3.43 (1H, m), 3.93 (2H, s), 4.17 (1H, sextet), 4.43-4.50 (1H, m), 7.15-7.30 (3H, m), 8.03 (1H, d), 12.60 (1H, s).

17c: —N-(cyclopropylmethyl)-1-[2-fluoro-5-[(4-oxo-5,6,7,8-tetrahydro-3H-phthalazin-1-yl)methyl]benzoyl]piperidine-4-carboxamide; ¹H NMR (399.902 MHz, DMSO) δ 0.13-0.17 (2H, m), 0.38-0.42 (2H, m), 0.84-0.94 (1H, m), 1.42-1.57 (2H, m), 1.59-1.68 (5H, m), 1.75-1.82 (1H, m), 2.36-2.44 (5H, m), 2.83 (1H, td), 2.95 (2H, t), 3.00-3.09 (1H, m), 3.38-3.44 (1H, m), 3.94 (2H, s), 4.44-4.52 (1H, m), 7.17-7.31 (3H, m), 7.88 (1H, t), 12.61 (1H, s).

17d: —1-[2-fluoro-5-[(4-oxo-5,6,7,8-tetrahydro-3H-phthalazin-1-yl)methyl]benzoyl]-N-(2-methylpropyl)piperidine-4-carboxamide; ¹H NMR (399.902 MHz, DMSO) δ 0.83 (6H, d), 1.41-1.57 (2H, m), 1.59-1.72 (6H, m), 1.75-1.81 (1H, m), 2.35-2.44 (5H, m), 2.77-2.89 (3H, m), 2.99-3.08 (1H, m), 3.37-3.44 (1H, m), 3.93 (2H, s), 4.44-4.50 (1H, m), 7.16-7.30 (3H, m), 7.79 (1H, t), 12.60 (1H, s).

17e: —4-[[4-fluoro-3-[4-(morpholine-4-carbonyl)piperidine-1-carbonyl]phenyl]methyl]-5,6,7,8-tetrahydro-2H-phthalazin-1-one; ¹H NMR (399.902 MHz, DMSO) δ 1.41-1.68 (7H, m), 1.71-1.78 (1H, m), 2.35-2.42 (4H, m), 2.84-2.97 (2H, m), 3.06-3.14 (1H, m), 3.36-3.61 (9H, m), 3.93 (2H, s), 4.45-4.51 (1H, m), 7.17-7.30 (3H, m), 12.61 (1H, s).

17f: —4-[[4-fluoro-3-[4-(2-methylpiperidine-1-carbonyl)piperidine-1-carbonyl]phenyl]methyl]-5,6,7,8-tetrahydro-2H-phthalazin-1-one; ¹H NMR (399.902 MHz, DMSO) complex NMR due to presumed rotamers.

17 g: —N-(2-dimethylaminoethyl)-1-[2-fluoro-5-[(4-oxo-5,6,7,8-tetrahydro-3H-phthalazin-1-yl)methyl]benzoyl]-N-methylpiperidine-4-carboxamide; ¹H NMR (399.902 MHz, DMSO) complex NMR.

17 h: —N-[1-[1-[2-fluoro-5-[(4-oxo-5,6,7,8-tetrahydro-3H-phthalazin-1-yl)methyl]benzoyl]piperidine-4-carbonyl]piperidin-4-yl]-N-methylcyclopropanesulfonamide; ¹H NMR (399.902 MHz, DMSO) complex NMR.

### Example 5

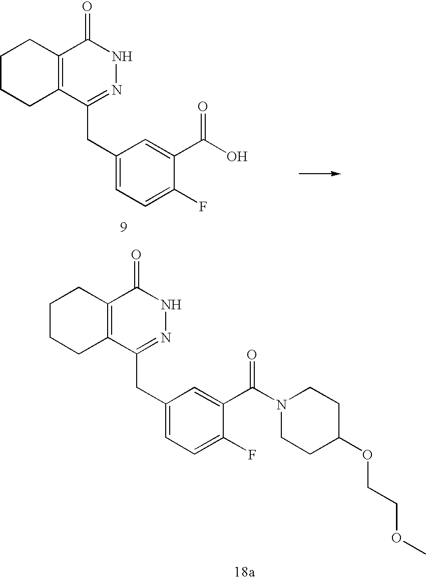

### (a) 4-(4-Fluoro-3-(4-(2-methoxyethoxy)piperidine-1-carbonyl)benzyl)-5,6,7,8-tetrahydrophthalazin-1(2H)-one (18a)

A solution of 2-fluoro-5-((4-oxo-3,4,5,6,7,8-hexahydrophthalazin-1-yl)methyl)benzoic acid (9) (153 mg, 0.51 mmol) in N,N-dimethylacetamide (4 mL) was treated with 4-(2-methoxyethoxy)piperidine hydrochloride (103 mg, 0.53 mmol) and triethylamine (0.212 mL, 1.52 mmol). O-Benzotriazol-1-yl-N,N,N′,N′-tetra-methyluronium hexafluorophosphate (253 mg, 0.67 mmol) was added and the resulting solution was stirred at ambient temperature for 3 hours. The crude reaction mixture was filtered and filtrate purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were evaporated to dryness and lyophilised to afford a gum, which was taken up in a small amount of diethyl ether and DCM and allowed to evaporate, before drying under vacuum, at 55° C., for 2 hours to afford the desired compound as a white foam (112 mg, 49.9% yield; 100% purity by LC-MS); ¹H NMR (400.132 MHz, DMSO) δ 1.30-1.50 (2H, m), 1.59-1.66 (4H, m), 1.72-1.79 (1H, m), 1.84-1.90 (1H, m), 2.35-2.40 (4H, m), 3.03-3.10 (1H, m), 3.25 (3H, s), 3.26-3.36 (2H, m), 3.44 (2H, t), 3.53-3.59 (3H, m), 3.90-4.00 (3H, m), 7.18-7.30 (3H, m), 12.60 (1H, s); m/z (LC-MS, ESI+), RT=1.46 (M+H 444.1).

### (b) Products Using Above Method (18b-e)

Using an analogous procedure to that described in (a), 2-fluoro-5-((4-oxo-3,4,5,6,7,8-hexahydrophthalazin-1-yl)methyl)benzoic acid (9) was reacted overnight with the appropriate piperidine to afford the compounds described below.

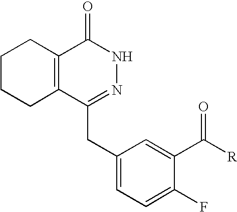

| | | Puri- | RT | |
|---|---|---|---|---|
| | R | ty | (min) | M + H |
| 18b | 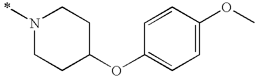 | 100 | 2.26 | 492.1 |
| 18c | 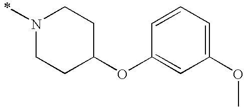 | 100 | 2.32 | 492.1 |
| 18d | 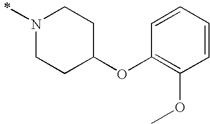 | 100 | 2.21 | 492.1 |
| 18e | 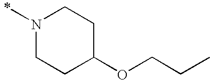 | 100 | 2.06 | 428.1 |

18b: —4-(4-fluoro-3-(4-(4-methoxyphenoxy)piperidine-1-carbonyl)benzyl)-5,6,7,8-tetrahydrophthalazin-1(2H)-one; ¹H NMR (400.132 MHz, DMSO) δ 1.48-1.66 (6H, m), 1.80-1.88 (1H, m), 1.92-2.00 (1H, m), 2.35-2.40 (4H, m), 3.14-3.20 (1H, m), 3.35-3.50 (2H, m), 3.70 (3H, s), 3.90-4.01 (3H, m), 4.47-4.52 (1H, m), 6.83-6.87 (2H, m), 6.91-6.95 (2H, m), 7.20-7.30 (3H, m), 12.60 (1H, s).

18c: —4-(4-fluoro-3-(4-(3-methoxyphenoxy)piperidine-1-carbonyl)benzyl)-5,6,7,8-tetrahydrophthalazin-1(2H)-one; ¹H NMR (400.132 MHz, DMSO) δ 1.49-1.68 (6H, m), 1.84-1.92 (1H, m), 1.96-2.04 (1H, m), 2.34-2.41 (4H, m), 3.16-3.25 (1H, m), 3.36-3.52 (2H, m), 3.73 (3H, s), 3.92 (2H, s), 3.94-4.03 (1H, m), 4.62-4.67 (1H, m), 6.50-6.59 (3H, m), 7.15-7.30 (4H, m), 12.60 (1H, s).

18d: —4-(4-fluoro-3-(4-(2-methoxyphenoxy)piperidine-1-carbonyl)benzyl)-5,6,7,8-tetrahydrophthalazin-1(2H)-one; ¹H NMR (400.132 MHz, DMSO) δ 1.52-1.69 (6H, m), 1.80-1.88 (1H, m), 1.92-2.00 (1H, m), 2.35-2.40 (4H, m), 3.13-3.21 (1H, m), 3.38-3.51 (2H, m), 3.76 (3H, s), 3.90-4.02 (3H, m), 4.49-4.54 (1H, m), 6.85-7.05 (4H, m), 7.20-7.31 (3H, m), 12.60 (1H, s).

18e: —4-(4-fluoro-3-(4-propoxypiperidine-1-carbonyl)benzyl)-5,6,7,8-tetrahydrophthalazin-1(2H)-one; ¹H NMR (400.132 MHz, DMSO) δ 0.87 (3H, t), 1.30-1.54 (4H, m), 1.57-1.66 (4H, m), 1.71-1.78 (1H, m), 1.83-1.90 (1H, m), 2.34-2.40 (4H, m), 3.03-3.11 (1H, m), 3.28-3.40 (4H, m), 3.49-3.55 (1H, m), 3.89-3.99 (3H, m), 7.17-7.29 (3H, m), 12.59 (1H, s).

### Example 6

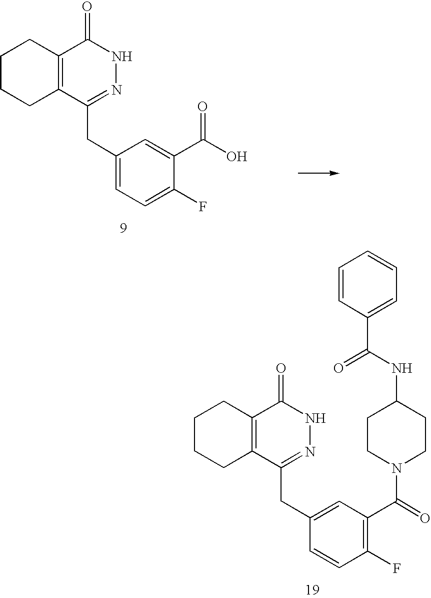

### (a) N-(1-(2-Fluoro-5-((4-oxo-3,4,5,6,7,8-hexahydrophthalazin-1-yl)methyl)benzoyl)piperidin-4-yl)benzamide (19)

A partial solution of 2-fluoro-5-((4-oxo-3,4,5,6,7,8-hexahydrophthalazin-1-yl)methyl)benzoic acid (9) (212 mg, 0.70 mmol) in N,N-dimethylacetamide (7 ml) was treated with N-Piperidin-4-yl-benzamide (157 mg, 0.77 mmol) and triethylamine (0.250 ml, 1.79 mmol). O-Benzotriazol-1-yl-N,N,N′,N′-tetra-methyluronium hexafluorophosphate (356 mg, 0.94 mmol) was then added and the reaction mixture was stirred at ambient temperature under nitrogen for 2 hours. The reaction mixture was filtered through a 0.45 μm syringe filter and the filtrate purified by preparative HPLC (Waters XBridge Prep C18 OBD column, 5μ silica, 19 mm diameter, 100 mm length), using decreasingly polar mixtures of water (containing 1% NH3) and MeCN as eluents. Fractions containing the desired compound were combined and further purified by preparative HPLC (Waters XBridge Prep C18 OBD column, 5μ silica, 19 mm diameter, 100 mm length), using decreasingly polar mixtures of water (containing 0.1% TFA) and MeCN as eluents. Fractions containing the desired compound were subjected to ion exchange chromatography, evaporated to dryness and lyophilised to afford the desired compound as a white solid (67.0 mg, 19.6% yield, 100% purity by LC-MS); ¹H NMR (400.132 MHz, DMSO) δ 1.40-1.66 (6H, m), 1.76-1.83 (1H, m), 1.88-1.95 (1H, m), 2.35-2.40 (4H, m), 2.93-3.00 (1H, m), 3.12-3.21 (1H, m), 3.38-3.45 (1H, m), 3.93 (2H, s), 4.04-4.14 (1H, m), 4.43-4.50 (1H, m), 7.16 (1H, dd), 7.22-7.32 (2H, m), 7.44-7.55 (3H, m), 7.82-7.86 (2H, m), 8.27-8.32 (1H, m), 12.61 (1H, s); m/z (LC-MS, ESI+), RT=1.88 (M+H 489.6).

### Example 7

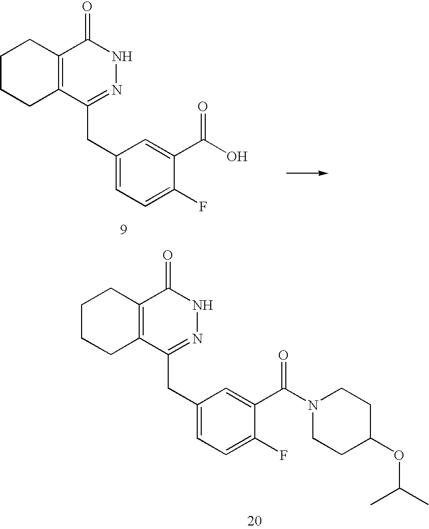

### (a) 4-(4-Fluoro-3-(4-isopropoxypiperidine-1-carbonyl)benzyl)-5,6,7,8-tetrahydrophthalazin-1(2H)-one (20)

A solution of 4-isopropoxypiperidine hydrochloride (119 mg, 0.66 mmol) and triethylamine (0.203 mL, 1.46 mmol) in DMF (2 mL) was added in one portion to a stirred solution of 2-fluoro-5-((4-oxo-3,4,5,6,7,8-hexahydrophthalazin-1-yl)methyl)benzoic acid (9) (200 mg, 0.66 mmol), triethylamine (0.203 mL, 1.46 mmol) and O-Benzotriazol-1-yl-N,N,N′,N′-tetra-methyluronium hexafluorophosphate (376 mg, 0.99 mmol) in DMF (2 mL) at ambient temperature. The resulting solution was stirred for 4 hours. The crude mixture was then purified by preparative HPLC (Waters XBridge Prep C18 OBD column, 5μ silica, 30 mm diameter, 100 mm length), using decreasingly polar mixtures of water (containing 1% NH3) and MeCN as eluents. Fractions containing the desired compound were evaporated to dryness and lyophilised to afford the desired compound as a gum (87 mg, 30.8% yield, 98.5% purity by LC-MS); ¹H NMR (399.902 MHz, DMSO) δ 1.08 (6H, dd), 1.26-1.46 (2H, m), 1.59-1.67 (6H, m), 1.68-1.75 (1H, m), 1.80-1.87 (1H, m), 2.32-2.43 (4H, m), 3.03-3.12 (1H, m), 3.25-3.29 (1H, m), 3.60-3.66 (1H, m), 3.70 (1H, quintet), 3.92 (2H, s), 7.19 (1H, dd), 7.23 (1H, d), 7.26-7.30 (1H, m), 12.61 (1H, s); m/z (LC-MS, ESI+), RT=1.89 (M+H 428.5).

### Example 8

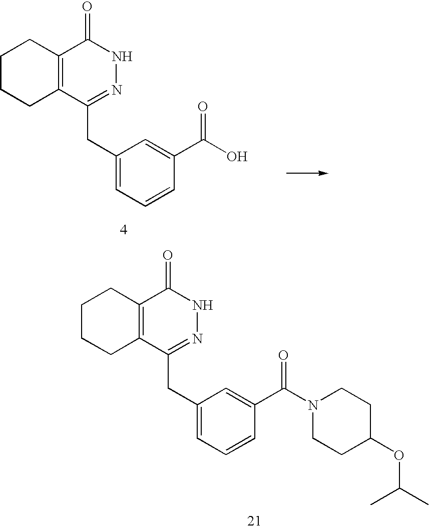

### (a) 4-(3-(4-isopropoxypiperidine-1-carbonyl)benzyl)-5,6,7,8-tetrahydrophthalazin-1(2H)-one (21)

A solution of 4-isopropoxypiperidine hydrochloride (126 mg, 0.70 mmol) and triethylamine (0.216 mL, 1.55 mmol) in DMF (2 mL) was added in one portion to a stirred solution of 3-((4-oxo-3,4,5,6,7,8-hexahydrophthalazin-1-yl)methyl)benzoic acid (4) (200 mg, 0.70 mmol), triethylamine (0.216 mL, 1.55 mmol) and O-Benzotriazol-1-yl-N,N,N′,N′-tetra-methyluronium hexafluorophosphate (400 mg, 1.06 mmol) in DMF (2 mL). The resulting solution was stirred at ambient temperature for 4 hours. The crude mixture was then purified by preparative HPLC (Waters XBridge Prep C18 OBD column, 5μ silica, 30 mm diameter, 100 mm length), using decreasingly polar mixtures of water (containing 1% NH3) and MeCN as eluents. Fractions containing the desired compound were evaporated to dryness and lyophilised to afford the desired compound as a gum (184 mg, 63.9% yield, 99.2% purity by LC-MS); ¹H NMR (399.902 MHz, DMSO) δ 1.08 (6H, t), 1.26-1.46 (2H, m), 1.58-1.65 (6H, m), 1.68-1.88 (2H, m), 2.33-2.42 (4H, m), 3.04-3.27 (2H, m), 3.59-3.65 (1H, m), 3.71 (1H, quintet), 3.95 (2H, s), 7.16-7.19 (1H, m), 7.25 (2H, dd), 7.38 (1H, t), 12.62 (1H, s); m/z (LC-MS, ESI+), RT=1.93 (M+H 410.6).

### Example 9

### Inhibitory Action

In order to assess the inhibitory action of the compounds, the following assay was used to determine IC₅₀ values.

Mammalian PARP, isolated from Hela cell nuclear extract, was incubated with Z-buffer (25 mM Hepes (Sigma); 12.5 mM MgCl₂ (Sigma); 50 mM KCl (Sigma); 1 mM DTT (Sigma); 10% Glycerol (Sigma) 0.001% NP-40 (Sigma); pH 7.4) in 96 well FlashPlates (TRADE MARK) (NEN, UK) and varying concentrations of said inhibitors added. All compounds were diluted in DMSO and gave final assay concentrations of between 10 and 0.01 μM, with the DMSO being at a final concentration of 1% per well. The total assay volume per well was 401.

After 10 minutes incubation at 30° C. the reactions were initiated by the addition of a 10 μl reaction mixture, containing NAD (5 μM), ³H-NAD and 30mer double stranded DNA-oligos. Designated positive and negative reaction wells were done in combination with compound wells (unknowns) in order to calculate % enzyme activities. The plates were then shaken for 2 minutes and incubated at 30° C. for 45 minutes.

Following the incubation, the reactions were quenched by the addition of 50 μl 30% acetic acid to each well. The plates were then shaken for 1 hour at room temperature.

The plates were transferred to a TopCount NXT (TRADE MARK) (Packard, UK) for scintillation counting. Values recorded are counts per minute (cpm) following a 30 second counting of each well.

The % enzyme activity for each compound is then calculated using the following equation:

$$\% Inhibition = 100 - ( 100 \times \frac{( cpm of unknowns - mean negative cpm )}{( mean positive cpm - mean neagative cpm )} )$$

IC₅₀ values (the concentration at which 50% of the enzyme activity is inhibited) were calculated, which are determined over a range of different concentrations, normally from 10 μM down to 0.001 μM. Such IC₅₀ values are used as comparative values to identify increased compound potencies.

All compounds tested had a mean IC₅₀ of less than 0.1 μM.

The mean IC₅₀ results for compounds of the invention are listed below:

| | Mean |
|---|---|
| | IC₅₀ (μM) |
| 5a | 0.0048 |
| 5b | 0.0052 |
| 5c | 0.0041 |
| 5d | 0.0035 |
| 5e | 0.0073 |
| 5f | 0.0055 |
| 5g | 0.0180 |
| 5h | 0.0058 |
| 5i | 0.003 |
| 5j | 0.003 |
| 5k | 0.005 |
| 5l | 0.006 |
| 10a | 0.0029 |
| 10b | 0.0073 |
| 10c | 0.0027 |
| 10d | 0.0033 |
| 10e | 0.0053 |
| 10f | 0.0032 |
| 10g | 0.0022 |
| 10h | 0.0046 |
| 10i | 0.0058 |
| 10j | 0.0042 |
| 10k | 0.0059 |
| 10l | 0.0054 |
| 10m | 0.052 |
| 10o | 0.004 |
| 10q | 0.0051 |
| 10r | 0.002 |
| 10s | 0.002 |
| 10t | 0.003 |
| 10u | 0.004 |
| 10v | 0.004 |
| 10x | 0.004 |
| 10z | 0.003 |
| 10aa | 0.004 |
| 10ab | 0.004 |
| 10ac | 0.006 |
| 10ad | 0.003 |
| 10ae | 0.006 |
| 10af | 0.003 |
| 14 | 0.005 |
| 17a | 0.003 |
| 17b | 0.002 |
| 17c | 0.007 |
| 17d | 0.006 |
| 17e | 0.007 |
| 17f | 0.004 |
| 17g | 0.009 |
| 17h | 0.003 |
| 18a | 0.005 |
| 18b | 0.005 |
| 18c | 0.006 |
| 18d | 0.002 |
| 18e | 0.003 |
| 19 | 0.005 |
| 20 | 0.003 |
| 21 | 0.008 |

### Potentiation Factor

The Potentiation Factor (PF₅₀) for compounds is calculated as a ratio of the IC₅₀ of control cell growth divided by the IC₅₀ of cell growth+PARP inhibitor. Growth inhibition curves for both control and compound treated cells are in the presence of the alkylating agent methyl methanesulfonate (MMS). The test compounds were used at a fixed concentration of 0.2 micromolar. The concentrations of MMS were over a range from 0 to 10 μg/ml.

Cell growth was assessed using the sulforhodamine B (SRB) assay (Skehan, P., et al., (1990) New colorimetric cytotoxicity assay for anticancer-drug screening. J. Natl. Cancer Inst. 82, 1107-1112.). 2,000 HeLa cells were seeded into each well of a flat-bottomed 96-well microtiter plate in a volume of 100 μl and incubated for 6 hours at 37° C. Cells were either replaced with media alone or with media containing PARP inhibitor at a final concentration of 30 nM or 200 nM. Cells were allowed to grow for a further 1 hour before the addition of MMS at a range of concentrations (typically 0, 1, 2, 3, 5, 7 and 10 μg/ml) to either untreated cells or PARP inhibitor treated cells. Cells treated with PARP inhibitor alone were used to assess the growth inhibition by the PARP inhibitor.

Cells were left for a further 16 hours before replacing the media and allowing the cells to grow for a further 72 hours at 37° C. The media was then removed and the cells fixed with 100 μl of ice cold 10% (w/v) trichloroacetic acid. The plates were incubated at 4° C. for 20 minutes and then washed four times with water. Each well of cells was then stained with 100 μl of 0.4% (w/v) SRB in 1% acetic acid for 20 minutes before washing four times with 1% acetic acid. Plates were then dried for 2 hours at room temperature. The dye from the stained cells was solubilized by the addition of 100 μl of 10 mM Tris Base into each well. Plates were gently shaken and left at room temperature for 30 minutes before measuring the optical density at 564 nM on a Microquant microtiter plate reader.

The following compounds had a mean PF₅₀ at 200 nM of at least 2: 5a, 5c-f, 5 h, 5k, 5l, 10a-j, 10l-10m, 10o, 10r, 10ab-10ae.

The following compounds had a mean PF₅₀ at 30 nM of at least 2: 5i-5k, 10o, 10q, 10s-x, 10z, 10aa, 14, 17c, 17d, 17f, 18a-e, 19, 20, 21.

### Solubility Assay

A typical assay that may be used to assess the solubility of the compounds of the present invention is as follows. The solubility of the compound is assessed in water and phosphate-buffered saline (pbs) at pH 7.4. The samples are all allowed to equilibrate in the solvent (with shaking) for 20 hours at room temperature. After that period, the samples will be visually examined to determine the presence/absence of un-dissolved solid. The samples will be centrifuged or filtered as necessary to remove insoluble material, and the solution analysed to determine solubility of the DS, diluting both aqueous and DMSO samples to a similar concentration with DMSO. The area of the peak obtained by HPLC (using the diode array detector) from the sample will be compared to the area of the peak from the DMSO solution (diluted to the same concentration as the sample) and quantified taking into account the weight of sample taken for initial dissolution. The assumption is made that the sample will be completely soluble in DMSO at the levels used for testing.

Comparing the ratio of the peak areas, and knowing the concentration of the original samples, the solubility may be calculated.

### Preparation of Samples

About 1 mg of the sample is weighed accurately into a 4-ml glass vial and exactly 1.0 ml of water, aqueous buffer or DMSO, is added to it by pipette. Each vial is ultrasonicated for up to 2 minutes to assist solubilisation of the solid. The samples are retained at room temperature for 20 hours, shaking on an orbital shaker. The vials are examined after this period to determine the presence/absence of un-dissolved solid. The samples should be centrifuged, or filtered through a 0.45 μm filter, to remove insoluble material if necessary, and the filtrate analysed to determine concentration of the compound in solution, after diluting all samples as appropriate with DMSO. 20 μl is injected onto the HPLC using the method shown below, injecting all samples in duplicate. The maximum solubility that can be determined using this method is nominally 1.0 mg/ml, the weight taken divided by the volume of solvent used.

### Analytical Techniques

The samples are subjected to LC/MS using a Waters Micromass ZQ instrument (or equivalent) with test parameters typically as follows.

Waters Micromass ZQ in positive ion mode.
Scanning from m/z 100 to 800
Mobile phase A—0.1% aqueous formic acid
Mobile phase B—0.1% formic acid in Acetonitrile
Column—Jones Chromatography Genesis 4μ C18 column, 4.6×50 mm
Flow rate 2.0 ml/min
Injection volume 30 μl injection into a 20 μl loop.
Gradient—starting at 95% A/5% B, rising to 95% B after 4 minutes, holding there for four minutes, then back to the starting conditions. (This may be modified if necessary to obtain better separation of peaks).
PDA detection scanning from 210 to 400 nm

### Quantification of Samples

Initial examination of the sample vials containing the aqueous dilution indicates whether or not the compound is soluble in that buffer at that concentration. If it is not soluble, this should be reflected in the concentration obtained in solution by HPLC/MS. If the solution is clear, then the concentration in aqueous solvent should be similar to that in DMSO, unless degradation of the compound has occurred; this should be visible on the chromatogram.

The assumption is made that the samples will be completely soluble in DMSO, therefore the peak size obtained from that sample will reflect 100% solubility. Assuming that the dilutions of all samples have been the same, then solubility in mg/ml=(area from pbs solution/area from DMSO solution)×(original weight in DMSO solution/dilution).

### Assay for Activity in Multidrug Resistant Cells

This assay measures the effectiveness of the test compounds in KBA1 cells, which are multidrug resistant Hela cells of cervical origin that express MDR1 (a P-glycoprotein which is an ATP dependent drug efflux pump responsible for decreased drug accumulation) and which are highly resistant to etoposide. In the assay these cells are matched with KB31 non-MDR1 expressing cells. This assay therefore examines the effect of MDR1 on the efficacy of tested compounds in KBA1 cells in comparison with KB31 cells which do not express MDR1. Verapamil is then used to reverse any MDR1 mediated effects in KBA1 cells.

### Method

100 μl of KBA1 Pgp expressing cells and/or KB31 matched non-Pgp expressing cells are seeded at 2×104/ml per well into 96 well tissue culture plate and left to adhere for 4-6 hours, which gives a final concentration of 2000 cells per well. Either 10 μL of Verapamil in cell media (giving final concentration of 10 μM) or 10 μl of normal media is then added to the wells, followed by incubation for 30 minutes at 37° C.

10 μl of the test compound is then added to give final concentrations of 50, 40, 30, 20, 10, and 5 μM. Etoposide (VP16) is used as a positive control. The KBA1 cells should be treated to give a final concentration of 2, 1, 0.5, 0.25, 0.1, 0.05 μg/ml and KB31 cells 0.25, 0.1, 0.05, 0.025, 0.01, 0.005 μg/ml to ensure adequate cell kill for both cell lines. The control wells are treated with media and the equivalent amount of DMSO, which should not exceed 1% of the final concentration. The resulting plates are incubated at 37° C. for 72 hours.

At the end of the incubation, the cells are washed with PBS then stained with SRB (sulforhodamineB) to give total protein levels, read on a UV/vis plate reader. The data can then be used to calculate the IC₅₀ of the test compounds in the KBA1 and KB31 cell lines, and these values compared to indicate the effect of MDR1 on the test compounds.

1: A compound of the formula (I): 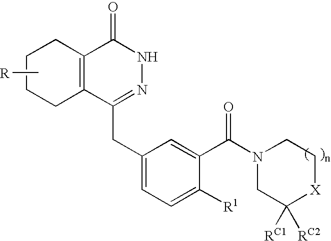
wherein:
R represents one or more optional substituents on the fused cyclohexene ring;
X can be NR^{X} or CR^{X}R^{Y};
if X=NR^{X} then n is 1 or 2 and if X=CR^{X}R^{Y} then n is 1;
if X=NR^{X}, then R^{X} is selected from the group consisting of H, optionally substituted C₁₋₂₀ alkyl, optionally substituted C₅₋₂₀ aryl, optionally substituted C₃₋₂₀ heterocyclyl, optionally substituted amido, optionally substituted thioamido, optionally substituted ester, optionally substituted acyl, and optionally substituted sulfonyl groups;
if X=CR^{X}R^{Y} then R^{X} is selected from the group consisting of H, optionally substituted C₁₋₂₀ alkyl, optionally substituted C₅₋₂₀ aryl, optionally substituted C₃₋₂₀ heterocyclyl, optionally substituted amido, optionally substituted thioamido, optionally substituted sulfonamino, optionally substituted ether, optionally substituted ester, optionally substituted acyl, optionally substituted acylamido, and optionally substituted sulfonyl groups and R^{Y} is selected from H, hydroxy, optionally substituted amino, or R^{X} and R^{Y} may together form an optionally substituted spiro-C₃₋₇ cycloalkyl or heterocyclyl group;
R^{C1} and R^{C2} are both hydrogen, or when X is CR^{X}R^{Y}, R^{C1}, R^{C2}, R^{X} and R^{Y}, together with the carbon atoms to which they are attached, may form an optionally substituted fused aromatic ring; and
R¹ is selected from H and halo.

2: The compound according to claim 1, which is of formula Id:

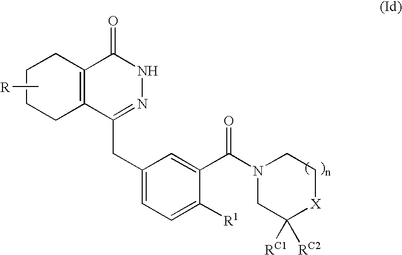

3: The compound according to claim 1, wherein R is selected from halo, nitro, hydroxy, ether, thiol, thioether, amino, C₁₋₇ alkyl, C₃₋₂₀ heterocyclyl and C₅₋₂₀ aryl.

4: The compound according to claim 1, wherein R¹ is selected from H, Cl and F.

5: The compound according claim 1, wherein R^{C1} and R^{C2} are both hydrogen.

6: The compound according to claim 1, wherein n is 2, X is NR^{X}, and R^{X} is selected from the group consisting of: H; optionally substituted C₁₋₂₀ alkyl; optionally substituted C₅₋₂₀ aryl; optionally substituted ester groups; optionally substituted acyl groups; optionally substituted amido groups; optionally substituted thioamido groups; and optionally substituted sulfonyl groups.

7: The compound according to claim 1, wherein n is 1, X is NR^{X}, and R^{X} is selected from the group consisting of: H; optionally substituted C₁₋₂₀ alkyl; optionally substituted C₅₋₂₀ aryl; optionally substituted acyl; and optionally substituted sulfonyl.

8: The compound according to claim 1, wherein n is 1, X is CR^{X}R^{Y}, R^{Y} is H, and R^{X} is selected from the group consisting of: H; optionally substituted C₃₋₂₀ heterocyclyl; optionally substituted amino; optionally substituted ester; and optionally substituted sulfonamino.

9: A pharmaceutical composition comprising a compound according to claim 1 and a pharmaceutically acceptable carrier or diluent.

10: A method of treating a disease ameliorated by the inhibition of PARP, comprising administering to a subject in need of treatment a therapeutically-effective amount of a compound according to claim 1.

11: The method according to claim 10, wherein the disease ameliorated by the inhibition of the activity of PARP is selected from: cancer, vascular disease; septic shock; ischaemic injury; neurotoxicity; haemorraghic shock; viral infection.

12: A method of treating of a patient with a cancer which is deficient in HR dependent DNA DSB repair activity, comprising administering to said patient a therapeutically-effective amount of a compound according to claim 1.
